(19) United States
(12) Patent Application Publication
(10) Pub. No.: US 2007/0010458 A1
(43) Pub. Date: Jan. 11, 2007

(54) Drugs for the arthritis treatment

(76) Inventor: Piero Del Soldato, Monza (IT)

Correspondence Address: ARENT FOX PLLC, 1050 CONNECTICUT AVENUE, N.W., SUITE 400, WASHINGTON, DC, 20036, US

(21) Appl. No.: 10/509,675

(22) PCT Filed: Mar. 27, 2003
(86) PCT No.: PCT/EP03/03183

§ 371 Date: Sep. 13, 2006

(30) Foreign Application Priority Data
Apr. 11, 2002 (IT) MI2002A000773

(51) Int. Cl. A61K 31/7048; A61K 31/7012; A61K 31/519; A61K 31/403; A61K 31/4035; A61K 31/375; A61K 31/353; A61K 31/343

(52) U.S. Cl. 514/23; 514/27; 514/456; 514/731; 514/469; 514/474; 514/690; 514/262.1; 514/546; 514/700; 514/411; 514/417; 514/438; 514/423; 514/291; 514/406; 514/378; 514/53

(57) ABSTRACT

Antiinflammatory and/or antiinflammatory/analgesic compounds having the following general formula:
A-(B)_{b0}—(C)_{c0}—N(O)ₛ (I) or salts thereof, wherein: A contains the radical of a non steroidal antiinflammatory or non steroidal antiinflammatory/analgesic drug, B and C are bivalent linking groups, are used in the arthritis therapy.

The present invention relates to the use of drugs for the arthritis therapy.

Arthritis pathological conditions are characterized by a progressive articulation damage due to the cartilaginoid matrix degradation. With arthritic diseases, it is generally meant diseases affecting articulations. Specifically rheumatoid arthrites, osteoarthrites, etc. can be mentioned.

The arthritis represents one of the most common medical problems and it is one of the main causes of disability. For example in the United States about 20 millions people result affected by arthritis. The factors which can cause the disease onset are various. Among these articulation traumas, obesity, or diseases modifying the cartilage structure or functionality, such for example rheumatoid arthritis, hemochromatosis, gout or Paget's disease, can be mentioned. Other factors are the age and sex. Generally the disease incidence is higher in women.

The arthritic process pathophysiology is progressive and the symptomatology is gradual and initially starts with the ache onset. The disease evolution determines damages to articulations, to tendons and can compromise leg/arm functionality.

The drugs used at present in the treament of arthritis are divided into two groups having different modes of action. The drugs of the first group, such as NSAIDs, provide symptomatic relief, but have no influence on the progress of the disease. The drugs belonging to the second group, have different chemical structures from the former and are effective on the course of the disease. For instance they can prevent irreversible joint damage. Said latter drugs are called disease-modifying agents. Presently the use in therapy of disease modifying agents is limited by their toxicity (Martindale, 31st Ed. 1996 pages 11-13).

At present specific therapies which intervene on the disease course reducing the degenerative effects on the cartilaginoid matrix, with side effects of small entity, so that the drugs can be used for the long term treatments which are generally required, do not exist.

The existing therapies are directed both to the ache treatment, administering analgesics such for example paracetamol, non steroidal antiinflammatory drugs (NSAIDs), and to the maintenance of the articulation functionality by the intraarticular application of drugs such for example corticosteroids or ialuronic acid, or parenteral such for example perdiacerine, sulfasalazine and penicillamine.

Among the above drugs used to treat the painful symptomatology, paracetamol is known to cause damages to liver and its assumption is contraindicated when other drugs are used. The NSAIDs cause even serious gastric damages and recent studies have shown that they can also accelerate the arthritic disease Rashad S., Lancet 1989, 519-522. The sulfasalazine can cause nausea, head-ache and skin rash. The penicillamine is bad tolerated and gives side effects, for example anorexia, nausea.

It is also known to use particular non steroidal antiinflammatory drugs having a 2-oxo-1H-indolic structure such, for example, Tenidap. This drug differently from the other NSAIDs is effective in arthritis interacting in the cytokine form ation, which are endogenous factors responsible for the inflammation and for the degradation of the cartilaginoid matrix. However Tenidap causes damages at hepatic and also renal level. See Martindale XXXIth Ed., pages 99-100.

Recently several studies have been directed to explain the arthritis etiopathology. These researches have shown that some inflammatory factors such for example cytokines, chemokines, etc. are involved in the activation of a cascade of catabolic and degenerative events determining the cartilaginoid matrix degradation.

It is known in the prior art that a group of growth factors, TGF-β proteins (TGF=transforming growth factor) in particular TGF-β1, play an important role in the articular cartilage reparation, promoting both the chondrocyte formation and the regeneration process of the bony tissue (osteoclastogenesis) (N. Felisaz et Al. Osteoarthritis and Cartilage (1999) 7 255 267).

The need was felt to have available compounds capable to induce the expression of the TGF-β proteins, so to be used in the arthritis treatment, without showing the side effects of the prior art drugs.

The Applicant has surprisingly and unexpectedly found compounds capable to solve the above technical problem.

An object of the invention is the use for the arthritis therapy as disease-modifying drugs of compounds or salts thereof having general formula:
A-(B)_{b0}—(C)_{c0}—N(O)ₛ (I)
wherein:
s is an integer and is equal to 1 or 2, preferably 2;
c0 is an integer and is 0 or 1;
b0 is an integer and is 0 or 1; with the proviso that at least one between c0 and b0 is different from zero;
A=R−T₁-, wherein
R— is the radical of a non steroidal antiinflammatory precursor drug excluding the compounds having 2-oxo-1H-indolic structure, or the radical of a non steroidal antiinflammatory/analgesic drug;
T₁=(CO)ₜ or (X)_{t′}, wherein X=—O—, —S—, —N(R_{1C})—, R_{1C} is H or a C₁-C₅ linear or branched alkyl, t and t′ are integers and equal to zero or 1, with the proviso that t=1 when t′=0; t=0 when t′=1;
   B=-T_{B}-X₂-T_{BI}- wherein
T_{B} and T_{BI} are equal or different;
T_{B}=(CO) when the reactive function in the precursor drug is —OH or —NH(R_{1C}); T_{B}=X, as above, when the reactive function in the precursor drug is —COOH;
T_{BI}=(CO)ₜₓ or (X)ₜₓₓ, wherein tx and txx have the value of 0 or 1; with the proviso that tx=1 when txx=0, tx=0 when txx=1; X is as above;
X₂ is a bivalent linking group as defined below;
   C is the bivalent radical -T_{C}-Y— wherein
when b0=c0=1: T_{C}=(CO) when tx=0, T_{C}=X when txx=0, X being as above;
when b0=0: T_{C}=(CO) when t=0, T_{C}=X when t′=0, X being as above;
when c0=0: tx=0, T_{BI}=X=—O—.
Y is:
Yₚ: 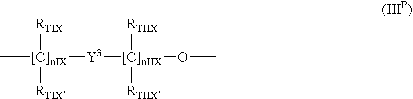
wherein:
nIX is an integer from 0 to 10, preferably from 1 to 3;
nIIX is an integer from 1 to 10, preferably from 1 to 3;
R_{TIX}, R_{TIX′}, R_{TIIX}, R_{TIIX′}, equal to or different from each other are H or C₁-C₄ linear or branched alkyl; preferably R_{TIX}, R_{TIX′}, R_{TIIX}, R_{TIIX′} are H.
Y³ is an heterocyclic saturated, unsaturated or aromatic ring, having 5 or 6 atoms, containing one or two nitrogen atoms, or Y can be:
Y₀, selected from the following:
   a —R′O— alkyleneoxy group wherein R′ is C₁-C₂₀ linear or branched when possible, preferably having from 2 to 6 carbon atoms or a cycloalkylene having from 5 to 7 carbon atoms, in the cycloalkylene ring one or more carbon atoms can be substituted by heteroatoms, the ring can have side chains of R′ type, R′ being as above; or one of the following groups: 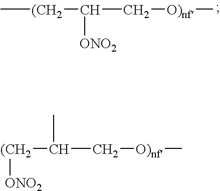
   wherein nf′ is an integer from 1 to 6 preferably from 1 to 4; 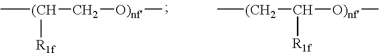
   wherein R_{1f}=H, CH₃ and nf′ is an integer from 1 to 6; preferably from 1 to 4;
or Y is Y_{Ar} and is selected from the following: 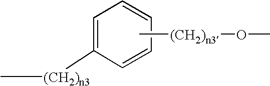
   wherein n3 is an integer from 0 to 3 and n3′ is an integer from 1 to 3; 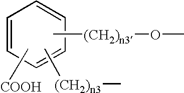
   wherein n3 and n3′ have the above meaning;
X₂, bivalent radical, is such that the corresponding precursor of B, -T_{B}-X₂-T_{BI}- wherein the free valences of T_{B} and of T_{BI} are each saturated with OZ, with Z or with —N(Z^{I}) (Z^{II}), wherein Z=H, C₁-C₁₀, preferably C₁-C₅ linear or branched when possible alkyl, Z^{I}, Z^{II} equal or different have the Z values as above, depending on that T_{B} and/or T_{BI}=CO or X, in function of the values of t, t′, tx and txx;
the precursor of B is selected from the following:
   aminoacids, preferably selected from the following: L-carnosine (formula CI), anserine (CII), selenocysteine (CIII), selenomethionine (CIV), penicillamine (CV), N-acetylpenicillamine (CVI), cysteine (CVII), N-acetylcysteine (CVIII), glutathione (CIX) or esters thereof, preferably ethyl or isopropyl ester: 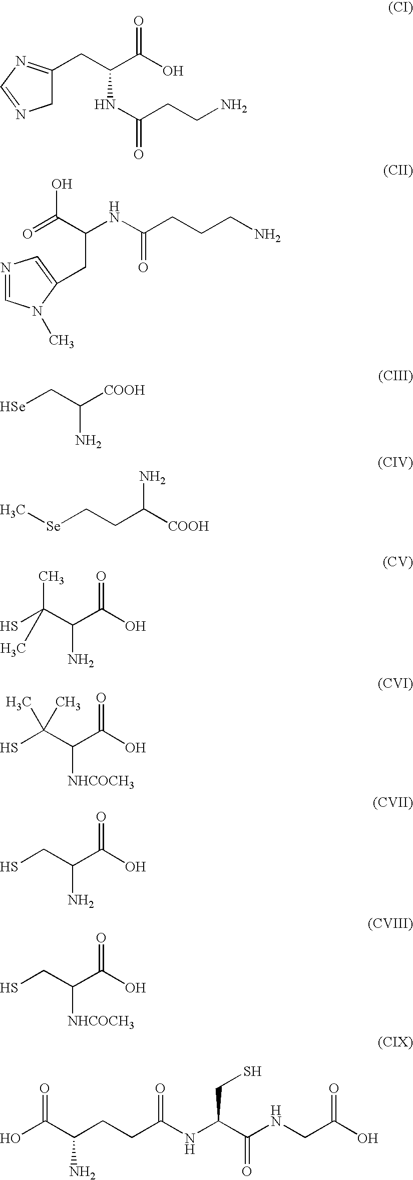
   hydroxyacids, preferably selected from the following: gallic acid (formula DI), ferulic acid (DII), gentisic acid (DIII), citric acid (DIV), caffeic acid (DV), dihydrocaffeic acid (DVI), p-cumaric acid (DVII), vanillic acid (DVIII): 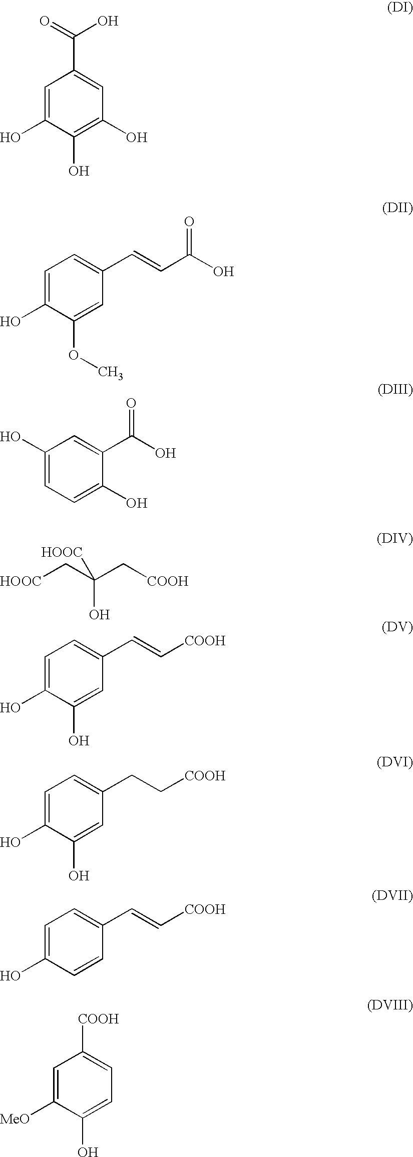
   aromatic and heterocyclic mono- and polyalcohols, preferably selected from the following: nordihydroguaiaretic acid (EI), quercetin (EII), catekin (EIII), kaempferol (EIV), sulphurethyne (EV), hydroquinone (EVIII), gossypol (EIX), reductic acid (EX), methoxyhydroquinone (EXI), hydroxyhydroquinone (EXII), propyl gallate (EXIII), 3,5-di-ter-butyl-4-hydroxybenzyl-thioglycolate (EXXIV), allopurinol (EXXXI); saccharose (EC), ascorbic (ECI) and isoascorbic acid (ECII), p-cumaric alcohol (ECIII), 4-hydroxyphenylethylalcohol (ECIV), coniferyl alcohol (ECV): 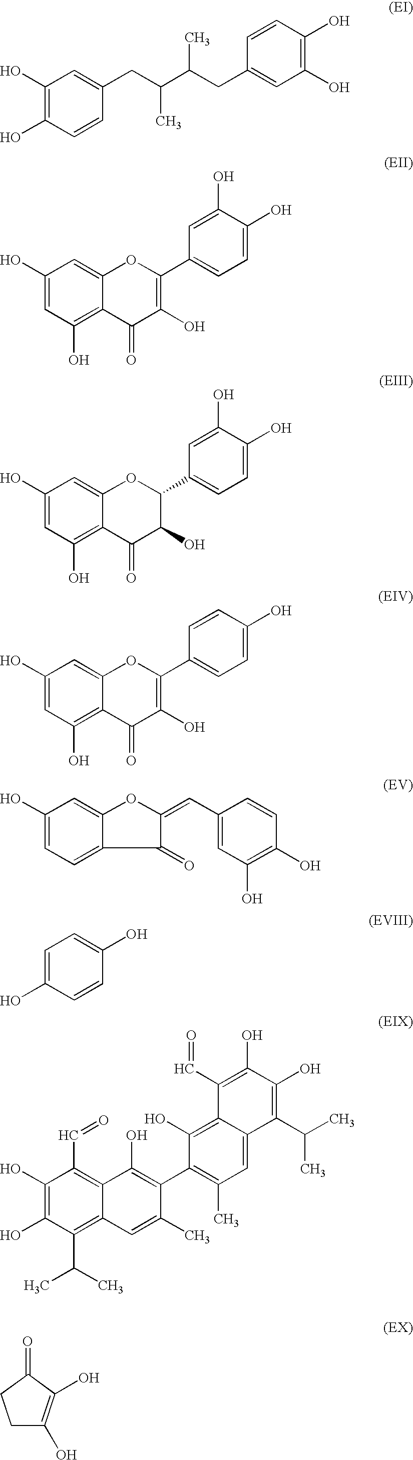 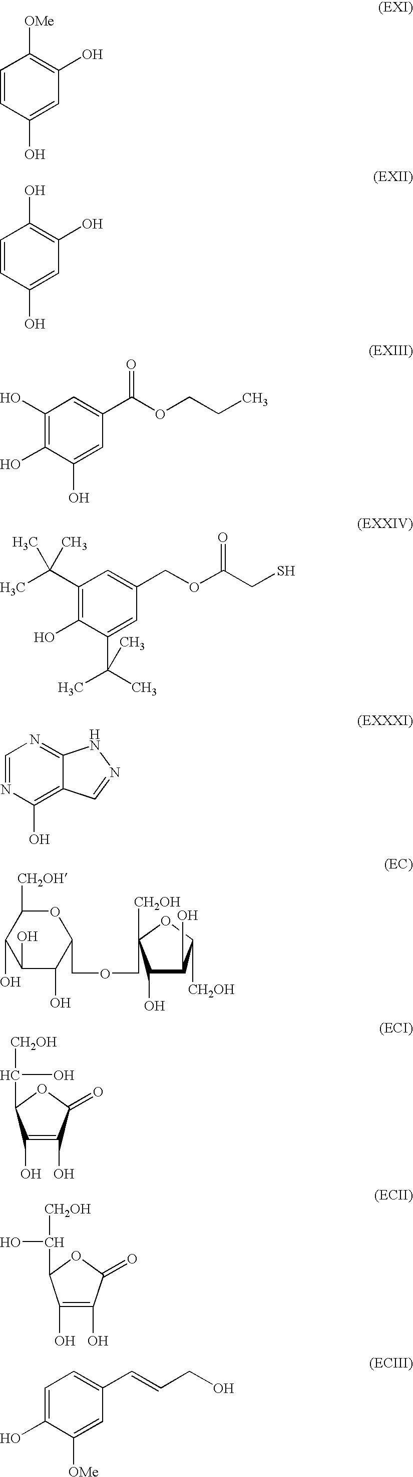 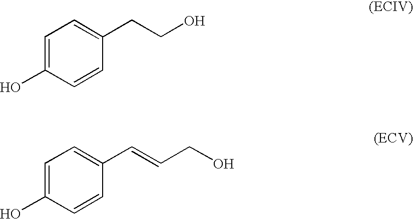
   compounds containing at least one free acid function, preferably selected from the following: 3,3′-thiodipropionic acid (NI), fumaric acid (NII), dihydroxymaleic acid (NIII), edetic acid (NV):

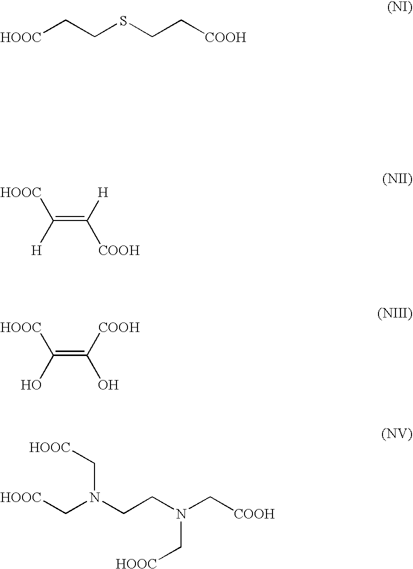

The compounds whose formulas have been indicated above are prepared according to known methods of the prior art, for example described in “The Merck Index”, 12a Ed. (1996), herein incorporated by reference. When available, the corresponding isomers and optical isomers can be used.

When b0=c0=1 the bonds between the drug radical and X₂ and between X₂ and Y can be, independently the one from the other, of ester, thioester, amide type; when b0=0 and c0=1 the bond between the drug radical and Y is of ester, thioester, amide type.

The radical R of non steroidal antiinflammatory drugs or antiinflammatory analgesic as above defined is selected from the following groups: 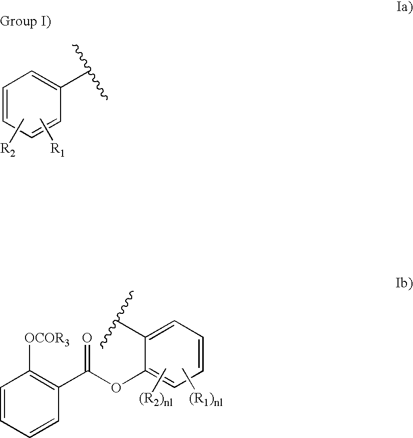
wherein:
R₁ is H or —OCOR₃; wherein R₃ is methyl, ethyl or C₃-C₅ linear or branched alkyl, or the residue of an heterocycle with only one ring having 5 or 6 atoms which can be aromatic, partially or totally hydrogenated, containing one or more heteroatoms independently selected from O, N and S;
R₂ is hydrogen, hydroxy, halogen, C₁-C₄ linear or branched when possible alkyl, C₁-C₄ linear or branched when possible alkoxyl; a C₁-C₄ linear or branched when possible perfluoroalkyl, for example trifluoromethyl; nitro, amino, mono- or di(C₁₋₄)alkylamino;
with the proviso that in formula Ia) R₁ and R₂ cannot be contemporaneously H, preferably when R₁=H R₂=OH;
preferably in the compounds of formula Ia) T₁=—CO— and:
—R₁=acetoxy, preferably in ortho position with respect to —CO—, R₂ is hydrogen; in this case the formula Ia) represents the acetylsalicylic acid residue;
R₁=H R₂=OH, preferably in ortho position with respect to —CO—, in this case the formula Ia) represents the salicylic acid residue;
   in formula Ib) nI is an integer 0 or 1;
   preferably in the compounds of formula Ib) R₃=CH₃, nI=0, T₁=—CO—; in this case Ib) is the acetylsalicylsalicylic acid residue; 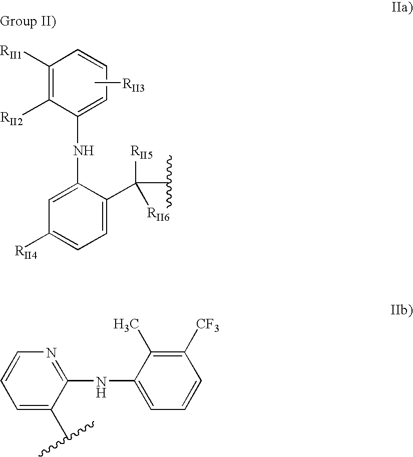
   wherein:
   R_{II5} is H, C₁-C₃ linear or branched when possible alkyl;
   R_{II6} has the same meaning of R_{II5}, or when R₁₁₅ is H it can be benzyl;
   R_{II1}, R_{II2} and R_{II3} can independently be hydrogen, C₁-C₆ linear or branched when possible alkyl, or C₁-C₆ linear or branched when possible alkoxy, or Cl, F, Br;
   R_{II4} is R_{II1} or bromine;
   the compounds wherein R_{II1}, R_{II4} are hydrogen and R_{II2} and R_{II3} are chlorine in ortho position with respect to NH are preferred; R_{II5} and R_{II6} are H, T₁=—CO—, when the free valence is saturated with OH the precursor compound is known as diclofenac. IIb) is the residue of the 2-[(2-methyl-3-(trifluoromethyl)phenyl]amino]-3-pyridincarboxylic] acid when T₁=—CO— and the free valence is saturated with OH the compound is known as flunixin;
   Group III) wherein R is: 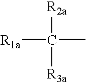
   wherein:
   R₂ₐ and R₃ₐ are H, C₁-C₁₂ linear or branched when possible alkyl or allyl, substituted or not, with the proviso that when one of the two is allyl, the other is H; preferably R₂ₐ and R₃ₐ, equal or different, are H, C₁-C₄ alkyl;
   R₁ₐ is selected from: 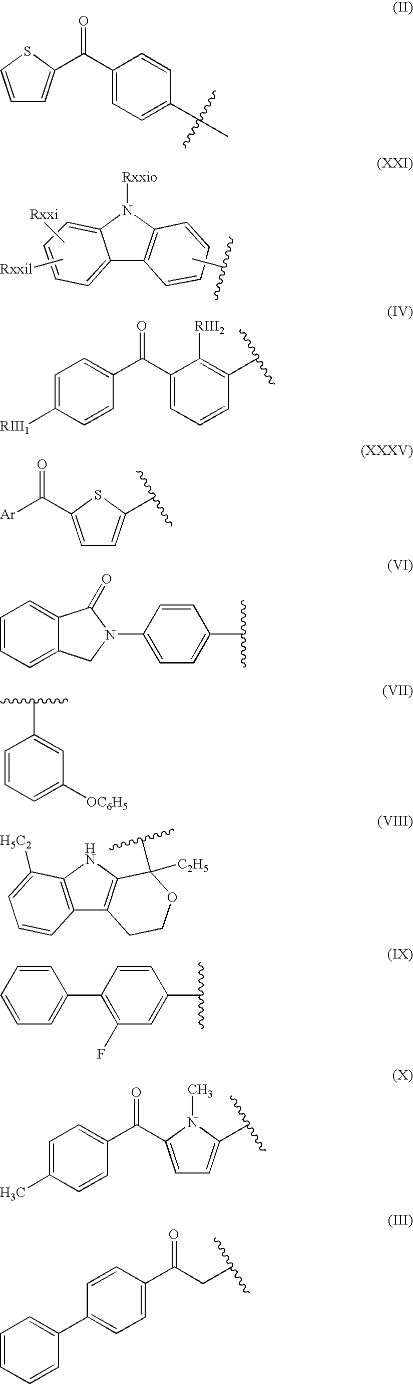
   IIID) R₁ₐ corresponds to the following formulas: 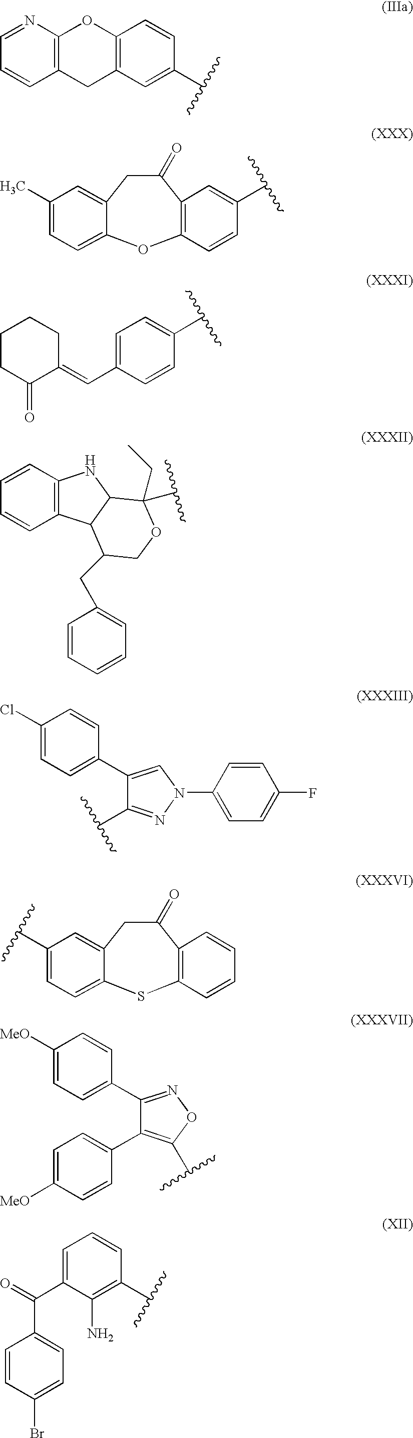 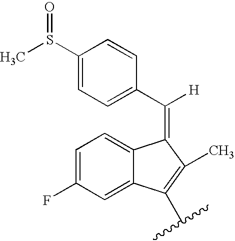
   wherein the meanings are the following:
when R₁ₐ is as defined in formula (IV), Ketoprofen residue:
R_{III1} is H, SR_{III3} wherein R_{III3} is C₁-C₄ linear or branched when possible alkyl;
R_{III2} is H, hydroxy;
the compounds are preferred wherein R_{III1} and R_{III2} are H, R₃ₐ is H, and R₂ₐ is methyl, T₁=—CO—;
when R₁ₐ is as defined in formula (XXI), carprofen residue:
Rₓₓᵢₒ is H, alkyl from 1 to 6 carbon atoms, linear or branched when possible, C₁-C₆ alkoxycarbonyl linked to a C₁-C₆ alkyl, C₁-C₆ carboxyalkyl, C₁-C₆ alkanoyl optionally substituted with halogens, benzyl or halobenzyl, benzoyl or halobenzoyl;
Rₓₓᵢ is H, halogen, hydroxy, CN, C₁-C₆ alkyl optionally containing OH groups, C₁-C₆ alkoxy, acetyl, benzyloxy, SRₓₓᵢ₂ wherein Rₓₓᵢ₂ is C₁-C₆ alkyl; C₁-C₃ perfluoroalkyl; C₁-C₆ carboxyalkyl optionally containing OH groups, NO₂, amino; sulphamoyl, di-alkyl sulphamoyl with C₁-C₆ alkyl or difluoroalkylsulphonyl with C₁-C₃ alkyl;
Rₓₓᵢ₁ is halogen, CN, C₁-C₆ alkyl containing one or more OH groups, C₁-C₆ alkoxy, acetyl, acetamido, benzyloxy, SR_{III3} being R_{III3} as above, C₁-C₃ perfluoroalkyl, hydroxy, C₁-C₆ carboxyalkyl, NO₂, amino, C₁-C₆ mono- or di-alkyl-amino; sulphamoyl, C₁-C₆ di-alkyl sulphamoyl, or di-fluoroalkylsulphamoyl as above; or Rₓₓᵢ together with Rₓₓᵢ₁ is a C₁-C₆ alkylene dioxy;
the compounds are preferred wherein Rₓₓᵢₒ is H, the linking group is in position 2, Rₓₓᵢ is H, Rₓₓᵢ₁ is chlorine and is in para position with respect to nitrogen;
R₃ₐ is H, R₂ₐ is methyl and T₁=—CO—;
when R_{1a′} is as defined in the formula (XXXV) tiaprofenic acid residue:
Ar is phenyl, hydroxyphenyl optionally mono or polysubstituted with halogen, alkanoyl and C₁-C₆ alkoxy, C₁-C₆ preferably C₁-C₃, trialkyl, cyclopentyl, cyclohexyl, cycloheptyl, heteroaryl, preferably thienyl, furyl optionally containing OH, pyridyl;
the preferred compounds of (XXXV) are those wherein Ar is phenyl, R₃ₐ is H, R₂ₐ is methyl and T₁=—CO—;
when R₁ₐ is as defined in formula (II), suprofen residue, of which that preferred has been indicated, in which R₃ₐ is H, R₂ₐ is methyl and T₁=—CO—, as described and obtained in U.S. Pat. No. 4,035,376 herein incorporated by reference;
when R₁ₐ is as defined in formula (VI), R is the residue of indoprofen when T₁=—CO—, R₂ₐ=H and R₃ₐ=CH₃; of indobufen when R₂ₐ is H and R₃ₐ=C₂H₅; T₁=—CO—, as described and obtained according to U.S. Pat. No. 3,997,669 herein incorporated by reference;
when R₁ₐ is as defined in formula (VIII), R is the etodolac residue when R₂ₐ=R₃ₐ=H and T₁=—CO—, as described and obtained according to U.S. Pat. No. 3,843,681 herein incorporated by reference;
when R₁ₐ is as defined in formula (VII), R is the fenoprofen residue when R₃ₐ=H, R₂ₐ=CH₃ and T₁=—CO—, as described and obtained according to U.S. Pat. No. 3,600,437 herein incorporated by reference;
when R₁ₐ is as defined in formula (III), R is the fenbufen residue when R₂ₐ=R₃ₐ=H and T₁=—CO—, as described and obtained according to U.S. Pat. No. 3,784,701 herein incorporated by reference;
when R₁ₐ is as defined in formula (IX), R is the flurbiprofen residue when R₃ₐ=H, R₂ₐ=CH₃, T₁=—CO—;
when R₁ₐ is as defined in formula (X) R is the tolmetin residue when R₂ₐ=R₃ₐ=H, T₁=—CO—, as described and obtained according to patent FR 1,574,570 herein incorporated by reference;
   In Group IIID) R₁ₐ corresponds to the following formulas:
IIIa), when R₂ₐ=H and R₃ₐ=CH₃ the pranoprofen residue is obtained: α-methyl-5H-[1]benzopyran-[2,3-b]pyridin-7-acetic acid; in the preferred compound R₂ₐ=H, R₃ₐ=CH₃, T₁=—CO— and in the precursor the free valence is saturated with OH;
(XXX), when R₂ₐ=H and R₃ₐ=CH₃ the bermoprofen residue is obtained: dibenz[b,f]oxepin-2-acetic acid; in the preferred compound R₂ₐ=H, R₃ₐ=CH₃, T₁=—CO—;
(XXXI), when R₂ₐ=H and R₃ₐ=CH₃, R is the radical of the compound CS-670: 2-[4-(2-oxo-1-cyclohexyliden methyl)phenyl]propionic acid; the preferred compound has R₂ₐ=H, R₃ₐ=CH₃, T₁=—CO—;
(XXXII), when R₂ₐ=R₃ₐ=H, the pemedolac residue is obtained; when R₂ₐ=R₃ₐ=H T₁=—CO—;
(XXXIII), when R_{2aa=R}₃ₐ=H, the pirazolac residue is obtained: 4-(4-chlorophenyl)-1-(4-fluorophenyl)-3-pyrazole acid derivatives;
the preferred compounds have R₂ₐ=R₃ₐ=H, T₁=—CO—;
(XXXVI), when R₂ₐ=H, R₃ₐ=CH₃ the zaltoprofen residue is obtained; when the residue is saturated with a hydroxyl or aminic group, or with the carboxylic function, the compounds are known as dibenzothiepine derivatives; in the preferred compounds R₂ₐ=H, R₃ₐ=CH₃, T₁=—CO—;
(XXXVII), when R₂ₐ=R₃ₐ=H the mofezolac residue is obtained: 3,4-di(p-methoxyphenyl)isoxazol-5-acetic acid when the residue is CH₂—COOH; in the preferred compounds R₂ₐ=R₃ₐ=H, T₁=—CO—;
(XII), when R₂ₐ=R₃ₐ=H the bromfenac residue is obtained: 2-amino-3-(4-bromobenzoyl)benzeneacetic acid; the preferred compounds have T₁=—CO—, R₂ₐ=R₃ₐ=H;
(XXXX) when R₂ₐ=R₃ₐ=H the sulindac residue is obtained: (Z)-5-fluoro-2-methyl-1-[[4-(methylsulphinyl)phenyl]-methylene]-1H-inden-3-acetic acid; the preferred compounds have T₁=—CO—, R₂ₐ=R₃ₐ=H;
   in group IV) R is 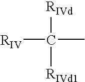
   wherein:
   R_{IVd} and R_{IVd1} are at least one H and the other an alkyl from C₁ to C₆ linear or branched when possible, preferably C₁-C₂, or difluoroalkyl with C₁-C₆ alkyl, C₁ preferred, or R_{IVd} and R_{IVd1} form together a methylene group;
   R_{IV} has the following meaning: 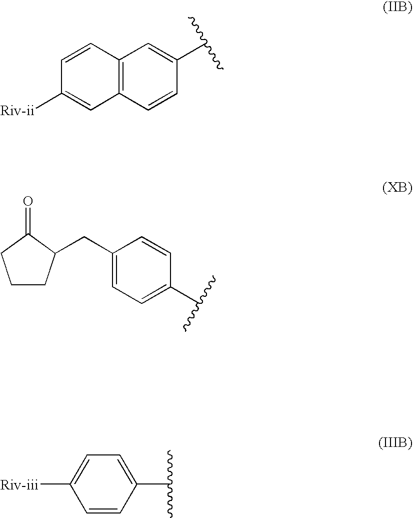
   wherein the compounds of group IV) have the following meaning:
in formula (IIB)
R_{iV-ii} is C₁-C₆ alkyl, C₃-C₇ cycloalkyl, C₁-C₇ alkoxymethyl, C₁-C₃ trifluoroalkyl, vinyl, ethynyl, halogen, C₁-C₆ alkoxy, difluoroalkoxy with C₁-C₇ alkyl, C₁-C₇ alkoxymethyloxy, alkylthiomethyloxy with C₁-C₇ alkyl, alkyl methylthio with C₁-C₇ alkyl, cyane, difluoromethylthio, phenyl- or phenylalkyl substituted with C₁-C₈ alkyl; preferably R_{iV-ii} is CH₃O—, R_{IVd} is H and R_{IVd1} is CH₃, and is known as naproxene residue; T₁=—CO—;
in formula (XB), of which the loxoprofen residue has been indicated, described in U.S. Pat. No. 4,161,538 herein incorporated by reference, the compounds are preferred wherein R_{IVd} is H and R_{IVd1} is CH₃; T₁=—CO—;
in formula (IIIB):
R_{iV-iii} is a C₂-C₅ alkyl, optionally branched when possible, C₂ and C₃ alkyloxy, allyloxy, phenoxy, phenylthio, cycloalkyl from 5 to 7 C atoms, optionally substituted in position 1 by a C₁-C₂ alkyl;
it is preferred the compound wherein R_{iV-iii} is 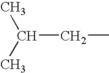
and R_{IVd}=H, R_{IVd1} is CH₃, compound known as ibuprofen residue, T₁=—CO—;
   Group V) 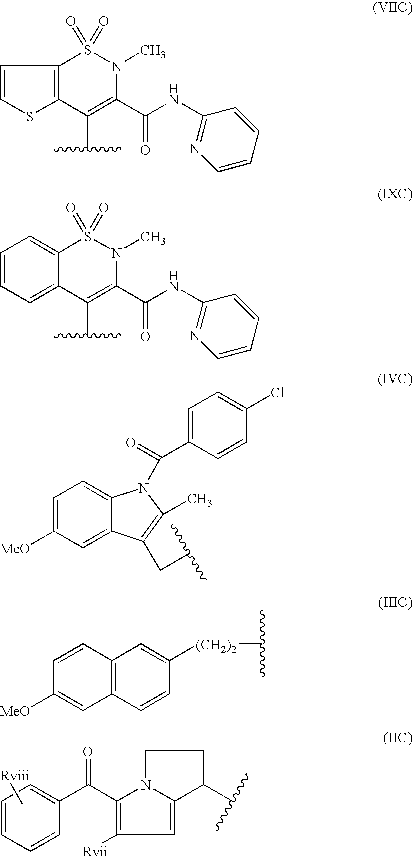
   Group VE) 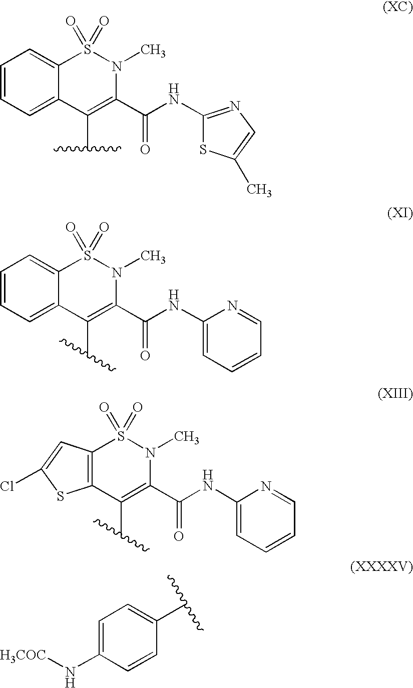
   In group V), the compounds have the following meanings:
when R is the formula (IIC),
R_{Vii} is H or a C₁-C₄ linear or branched when possible alkyl;
R_{Vii-1} is R_{Vii}, or C₁-C₄ linear or branched when possible alkoxy; Cl, F, Br; the position of R_{Vii-1} being ortho, or meta, or para;
the Ketorolac residue is preferred, wherein R_{Vii} and R_{Vii-1} are H, and T₁=—CO—;
when R is the formula (VIIC),
of which the tenoxicam residue has been indicated, T₁=—O—, as described and obtained in patent DE 2,537,070 herein incorporated by reference;
when R is the formula (IXC),
wherein T₁=—O—, the piroxicam residue has been indicated, as described and obtained in U.S. Pat. No. 3,591,584 herein incorporated by reference;
when R is the formula (IIIC)
wherein T₁=—CO—, of which the nabumetone residue has been indicated, as described and obtained in U.S. Pat. No. 4,061,779 herein incorporated by reference;
when R is the formula (IVC)
wherein T₁=—CO—, of which the indomethacin residue has been indicated, as described and obtained in U.S. Pat. No. 3,161,654 herein incorporated by reference;
when R is the formula (XC), the residue X is known as meloxicam; the preferred compounds are those wherein T₁=—CO—;
when R is the formula (XI) the residue is known as ampiroxicam when the termination is —CH(CH₃)OCOC₂H₅; the preferred compounds have T₁=—CO—;
when R is the formula (XIII) and the valence is saturated with H, the residue derives from lornoxicam; the preferred compounds have T₁=—O—;
when R is the formula (XXXXV), T₁=—O— and the valence is saturated with H, the compound known as paracetamol is obtained, as described and obtained in U.S. Pat. No. 2,998,450 herein incorporated by reference.

The compounds of formula (I) can be obtained as described in WO 95/30641, WO 00/61537, WO 01/12584.

Preferably Y³ is selected from the following bivalent radicals:

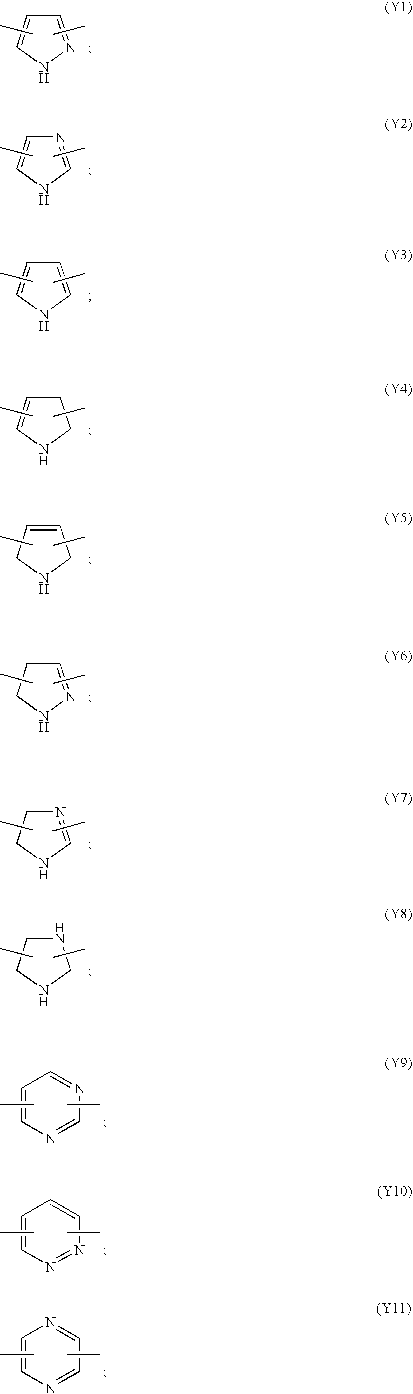

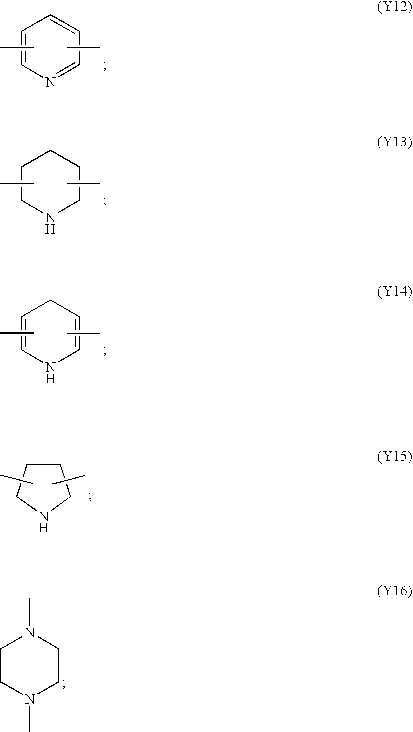

Preferred of Y³ are the following: (Y12), having the two free valences in the ortho positions with respect to the nitrogen atom; (Y16) with the two valences linked to the two heteroatoms; Y1 (pyrazol) 3,5-disubstituted; Y16 is particularly preferred.

The compounds according to the present invention, when at least one functional group salifiable with acids, for example an aminic group, is present, can be transformed into the corresponding salts. For example one way to form the salts is the following: when one basic nitrogen atom is present in the molecule, it is reacted in an organic solvent such for example acetonitrile, tetrahydrofuran with an equimolecular amount of the corresponding organic or inorganic acid.

Examples of organic acids are: oxalic, tartaric, maleic, succinic, citric, trifluoroacetic acids.

Examples of inorganic acids are: nitric, hydrochloric, sulphuric, phosphoric acids.

When the precursor compounds usable in the present invention have one or more chiral centres, they can be in racemic form or as diastereoisomer mixtures, as single enantiomers or single diastereoisomers; if they show a geometric asymmetry the compounds can be used in the cis or trans form.

The compounds of the present invention are prepared in the corresponding pharmaceutical compositions, even at belated release, for parenteral, oral and topical use, such for example sublingual, inhalatory, suppository, transdermal, enema, according to the well known techniques in the field, together with the usual excipients; see for example the volume “Remington's Pharmaceutical Sciences 15th Ed.”

The amount on a molar basis of the active principle in these compositions is generally the same, or lower, compared with that of the corresponding precursor drug.

The daily administrable doses are those of the precursor drugs, or optionally lower. The daily precursor doses can be found in the publications of the field, such for example “Physician's Desk Reference”.

Among the invention compounds those preferred are the following:
2-acetyloxybenzoic acid 3-nitrooxymethyl phenyl ester (I^{C});
2-fluoro-alpha-methyl[1,1′-biphenyl]-4-acetic acid 4-nitrooxy butylester (II^{C});
2-[(2,6-dichlorophenyl)amino]benzenacetic acid 4-nitrooxy butyl ester (III^{C});
(S)-N-acetyl-[alpha-methyl-4-(2-methylpropyl)benzenacetyl]cysteine 4-nitrooxybutylester having formula: 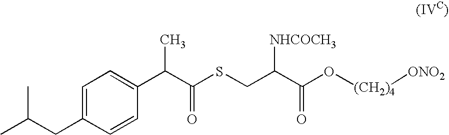
4-nitrooxybutanoic acid 4-acetylaminophenylester (V^{C});
trans-3-[4-[2-fluoro-alpha-methyl(1,1′-biphenyl)-4-acetyl oxy]-3-methoxyphenyl]-2-propenoic acid 4-(nitrooxy)butyl ester, having formula: 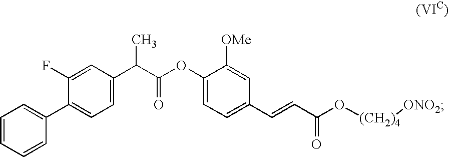
2-Fluoro-alpha-methyl[1,1′-biphenyl]-4-acetic acid 3-(nitrooxy methyl)phenyl ester having formula: 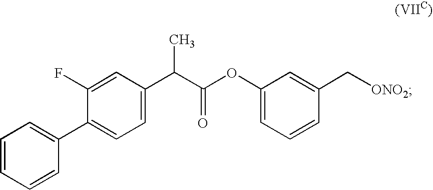
(S)-N-acetyl-[2-fluoro-alpha-methyl(1,1′-biphenyl)-4-acetyl]cysteine 4-(nitrooxy)butyl ester having formula: 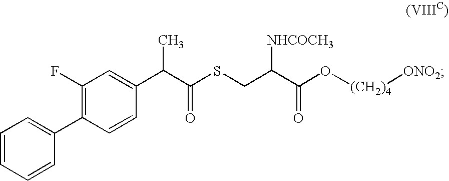
2-Fluoro-alpha-methyl[1,1′-biphenyl]-4-acetic acid 6-(nitrooxy methyl)-2-methylpyridyl ester having formula: 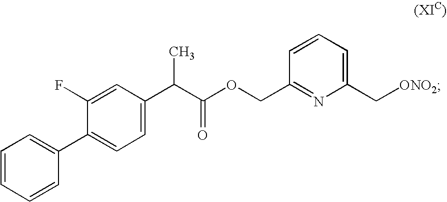
(S)-6-methoxy-alpha-methyl-2-naphthalenacetic acid 4-(nitrooxy) butyl ester having formula: 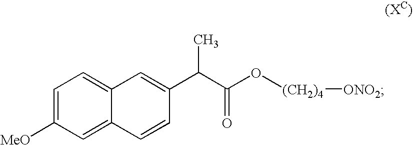
(S)-6-methoxy-alpha-methyl-2-naphthalenacetic acid 3-(nitrooxy methyl)phenyl ester having formula: 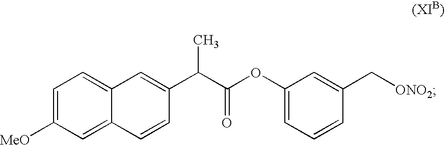
(S)-6-methoxy-alpha-methyl-2-naphthalenacetic acid 6-(nitro oxymethyl)-2-methylpyridyl ester having formula: 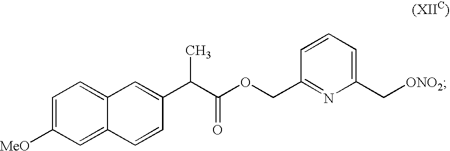
trans-3-[4-[6-methoxy-alpha-methyl-2-naphthalenacetyl oxy]-3-methoxyphenyl]-2-propenoic acid 4-(nitrooxy)butyl ester having formula: 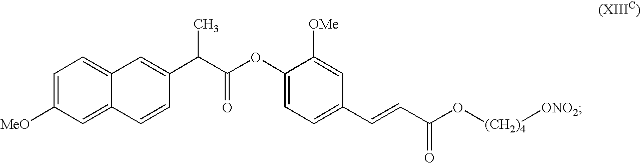
(S,S)-N-acetyl-S-(6-methoxy-alpha-methyl-2-naphthaleneacetyl) cysteine 4-(nitrooxy)butyl ester having formula: 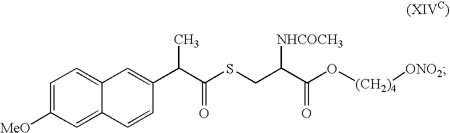
2-[(2,6-dichlorophenyl)amino]benzenacetic acid 4-(nitrooxy methyl)phenylmethyl ester having formula: 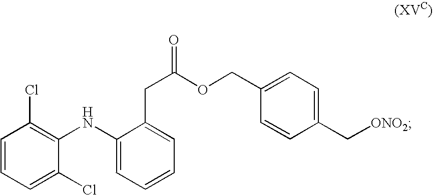
2-[(2,6-dichlorophenyl)amino]benzenacetic acid 6-(nitro oxymethyl)-2-methylpyridyl hydrochloride ester having formula: 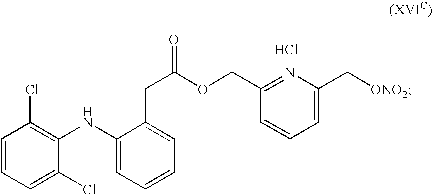
(S)-3-benzoyl-alpha-methyl-benzenacetic acid 4-(nitrooxy butyl)ester having formula: 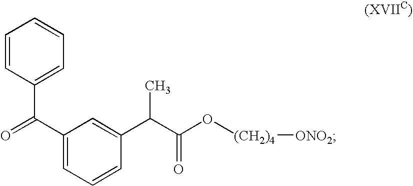
(S)-3-benzoyl-alpha-methyl-benzenacetic acid 3-(nitrooxy propyl)ester having formula: 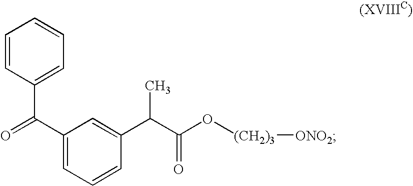
(S)-3-benzoyl-alpha-methyl-benzenacetic acid 4-(nitrooxy methyl)phenylmethyl ester having formula: 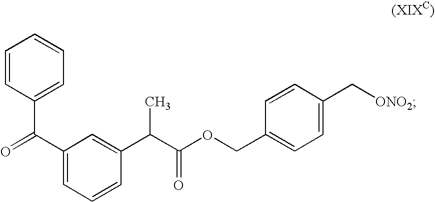
5-benzoyl-2,3-dihydro-1H-pyrrolizine-1-carboxylic acid 4-(nitrooxy)butyl ester having formula: 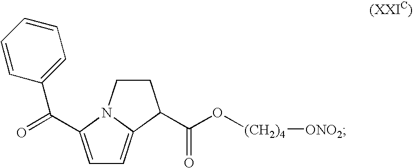
2-[(2,6-dichlorophenyl)amino]benzenacetic acid 5 (nitrooxy) ethyloxyethyl ester having formula: 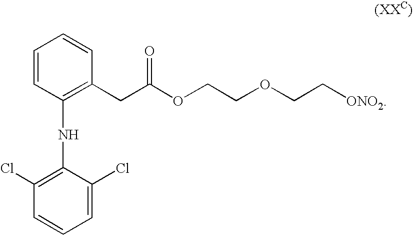
1-(4-Chlorobenzoyl)-5-methoxy-2-methyl-1H-indole-3-acetic acid 3-(nitrooxymethyl)phenyl ester (XXI^{C}).

It is surprising that the invention compounds are capable to promote the formation of the TGF-beta growth factor since it is known that the corresponding precursor compounds have no efficacy in reducing or preventing the cartilage degeneration process in the arthritic disease. Besides the Applicant has found that the NSAIDS precursor compounds have no effect on the formation of said growth factors.

Furthermore the present invention compounds have no side effects at gastric level and show an improved hepatic tolerability compared with the precursors. As an example, the Applicant has shown that the paracetamol nitroxybutylester has much more limited effects on the transaminase and bilirubin plasmatic levels compared with the paracetamol precursor.

Therefore the present invention compounds can be used in the arthritis therapy to prevent the cartilaginoid matrix degeneration, i.e. as curative and not only symptomatic drugs, combined with improved general tolerability.

The present invention compounds can be used also in the bony metabolism disease therapy, for example growth illness, characterized by an accelerated loss of the bony tissue, such as for example in old people.

It is known that the progressing of arthritic disease is due to the imbalance between pro-inflammatory (like IL-6, TNF-α) and anti-inflammatory (like TGF-β for example) mediators in different cells involved in the inflammation process, like monocytes, lymphocytes, chondrocytes, etc.

IL-6 (interleukin-6) is a potent pro-inflammatory cytokine and has been recognized to be implicated in rheumatoid arthritis (Choy E. H. et al., Arthritis Rheum. 46, 3143, 2002).

TNFα (Tumor necrosis factor α) has been shown to exert inflammatory changes in chondrocytes, such as decreased cell proliferation and decreased proteogycan synthesis. Overall these effects can be considered as signs of cartilage degradation and be implicated in the pathogenesis of arthritis.

Thus the effectiveness of a compound to inhibit TNFα induced-inflammatory changes in chondrocytes can be considered as a measure of the activity on arthritis, since the pharmacological action is to maintain the cartilage matrix integrity.

The compounds of the present invention are effective in reducing or eliminating the imbalance above said. They increase the formation of the anti-inflammatory mediators and decrease of the production of pro-inflammatory mediators.

Thus they have a more favourable pharmacotherapeutic profile than single cytokine-neutralizing agents (anti-TNF, etc) that must be given at very high doses, thus resulting in toxicity.

In rheumatoid arthritis disease a vast majority of patients have intermittent relapses and remissions of the disease. Unlike conventional NSAIDs administration of the drugs of the present invention can prevent disease relapses.

The following Examples are for illustrative purposes and are not limitative of the invention.

### EXAMPLE F1

Chondrocytes have been isolated from calf cartilage as described in Benya P. D., Biochemistry 1977; 16; 865-872, and used as primary cultures. The primary cultures have been kept in a DMEM culture medium (Dulbecco's modified Eagle medium) (high glucose) containing bovine fetal serum (10% vol.) and antibiotics at 37° C. and in air/CO₂ atmosphere (95%/5% vol.) until reaching the culture confluence. A cell sample is kept as a control and not treated with the tested compounds. The tested compounds are added to the other cellular cultures at the concentration 10⁻⁵ M and the so treated cultures have been incubated for 24 hours. The compounds have been previously dissolved in a DMSO amount such that the final concentration in the medium is 0.1%. The control has been treated only with DMSO.

The used compounds have been the following:
2-acetyloxybenzoic acid 3-nitrooxymethyl phenyl ester (NO-aspirin) prepared as described in Example 3 of WO 97/16405.
2-fluoro-alpha-methyl[1,1′-biphenyl]-4-acetic acid 4-nitrooxybutylester (NO-flurbiprofen), prepared as describe in Example 1 of WO 94/12463.
2-[(2,6-dichlorophenyl)amino]benzenacetic acid 4-nitrooxybutyl ester (NO-diclofenac), prepared as described in Example 1 of WO 94/04484.
(S)-N-acetyl-[alpha-methyl-4-(2-methylpropyl)benzenacetyl]cysteine 4-nitrooxybutylester (NO-ibuprofen), prepared as described in Example 2 of WO 00/6137.
4-nitrooxybutanoic acid 4-acetylaminophenylester (NO-paracetamol), prepared as described in Example 1 of WO 01/12584.

The following precursor compounds have been contemporaneously tested: aspirin and flurbiprofen.

At the end the cells have been washed 3 times with a medium free from serum and added with BSA (bovine serum albumin, 200 μg/ml) for 5, 30 and 60 minutes respectively and then incubated in a medium devoid of serum (1 ml) for further 6 hours. The conditioned medium has been collected, centrifuged and kept at −70° C. until the use.

Before the experiment, 0.5 ml of cellular culture supernatant have been acidified with HCl (0.1 ml, 1 N) and incubated at room temperature for 10 min, then neutralized with NaOH/HEPES (0.1 ml NaOH 1.2N/0.5 M).

CCL-64 cellular cultures lines in a proliferative state have been prepared as described in Jennings J. C., J. Cell. Physiol. 1988, 137, 167-72, sowing 2×10⁴ cells/well and incubating in the presence of FCS-medium (10% vol.).

After 24 hours the cells have been washed with the medium free from serum and incubated for 24 hours, respectively, with 0.5 ml of conditioned condrocyte medium, prepared as above and with increasing concentrations of TGF-β1 to determine a cellular growth inhibition reference curve, since the growth of said cellular lines is inhibited by the presence of TGF-β1.

At the twentieth hour ³H-timidine (0.5 μCi/ml), a cellular proliferation marker, which is incorporated in the DNA of the new cells has been added to the cultures. The cultures have then been incubated for 4 hours.

At the end the cells have been cold fixed (5° C.) with trichloroacetic acid 5% v/v, washed with the same solution and dissolved in NaOH (0.1 N). On the cells the count in liquid scintigraphy has been carried out to measure the marked timidine incorporated in the samples and in the standards treated with increasing amounts of TGF-β1. From the amount of incorporated timidine it is shown the amount of TGF-β1. The data reported in Table 1 are expressed in percentage of TGFβ1 produced in the samples treated with the tested compounds, compared with the untreated control. The data show that the tested compounds induce in the chondrocytes a significant increase of the TGFβ1 production compared with the untreated controls and the precursor compounds, and that the present invention compounds can therefore be used to prevent or reduce the articular tissue degradation.

### EXAMPLE F2

Hepatic Tolerability of Paracetamol v. the Corresponding Nitrooxybutylester (NO Paracetamol)

The nitrooxybutylester of paracetamol (NO-paracetamol) has been prepared as decribed in Example F1.

Groups of No. 10 rats have been treated i.p. with NO-paracetamol (1.4 g/Kg i.p.) or with paracetamol (1.16 g/Kg) or with the carrier (0.9% w/v NaCl containing 20% v/v di tween-20) (control group).

After 6 hours from the administration, the animals have been sacrificed, the blood has been collected and the plasma analyzed to determine the aspartate aminotransferase (AST) and alanine aminotransferase (ALT) and bilirubin concentrations. The results are reported in Table 2 and have been expressed in pecentage with respect to the values obtained in the control group (100%).

The results show that the paracetamol administration causes hepatic damage since there is an increase of the transaminase and bilirubin values with respect to the controls.

The NO-paracetamol administration does not cause ALT increase while the AST and bilirubin plasmatic levels are much lower than those of the groups treated with paracetamol, and as order of magnitude comparable with those of the controls.

### EXAMPLE F3

Effect of NO-Flurbiprofen and of Flurbiprofen on Interleukin (IL)-6 Release in Human Monocytes (Ex-Vivo Study)

IL-6 is a potent pro-inflammatory cytokine and has been recognized to be implicated in rheumatoid arthritis (Choy E. H. et al., Arthritis Rheum. 46, 3143, 2002).

Twenty-four healthy subjects of both sexes were enrolled and randomised into three groups of 8 subjects each. Each group was administered as it follows:
placebo: vehicle (0.5% aqueous suspension of carboxymethyl cellulose);
flurbiprofen: 100 mg twice a day;
NO-flurbiprofen: 100 mg twice a day; the compound was prepared as described in example F1.
   The treatment lasted seven consecutive days (oral subacute treatment).

Monocytes from whole blood samples obtained before and 4 hours after the last treatment were prepared. Monocytes were extracted by positive selection using paramagnetic beads loaded with anti-CD11 antibody. Cells were then incubated with 10 μg/ml endotoxin for 24 hours, and IL-6 released in cell supernatant measured by ELISA assay.

Results are reported in Table 3. Results are given as % in the confront of IL-6 release obtained in the placebo group.

The Table shows that oral subacute treatment of NO-flurbiprofen, but not of flurbiprofen, markedly suppressed IL-6 release in monocytes

### EXAMPLE F4

Effect of Flurbiprofen, NO-Flurbiprofen, Indomethacin, NO-Indomethacin (Indomethacin (3-Nitrooxymethyl)Phenyl Ester) on Interleukin (IL)-6 and TGF-β Release in Mouse Spleen Lymphocytes (In Vitro Study)

Spleen lymphocytes were prepared as it follows. Mice were killed by an overdose of ether, and spleens were collected and maintained in a sterile RPMI medium (Sigma-Aldrich) containing 0.5% (vol/vol) L-glutamine and 0.5% (vol/vol) sterile endotoxin-free fetal calf serum (FCS). The spleens were opened and the content (whole cells) collected and diluted with RPMI.

After repeated washings, cells were suspended in 10 ml of RPMI containing 1% (vol/vol) streptomycin and 1% (vol/vol) penicillin. The suspension was then incubated at 37° C. for 24 hours, in an O₂/CO₂ atmosphere (95%/5% v/v). Monocytes were eliminated by adhesion, and lysis of red cells was obtained by suspension in a solution 0.15 mol/liter NH₄Cl and 1 mmol/liter KHCO₃. The resulting lymphocytes were resuspended in RPMI-FCS, incubated for 30 minutes at 37° C. with anti-FAS, anti-FASL, or anti-IL₂ receptor monoclonal antibodies, and then washed twice with RPMI-FCS. Cells were then incubated with the FITC-conjugated secondary antibody for 30 mins at 4° C., washed twice, and resuspended in PBS/formaldehyde (0.5%). Control samples were treated with the FITC-conjugated secondary antibody only. Stained cells were analysed on a flow cytofluorimeter. Cells were gated using forward vs side scatter to exclude dead cells and debris.

Cells were transferred in plate and then 10 μg/ml endotoxin and each of the following compounds at a concentration of 50 μM added:
Placebo (no compound added);
Flurbiprofen;
NO-Flurbiprofen; the compound was prepared as described in ex. F1, above;
Indomethacin;
NO-indomethacin; the compound was prepared as described in the example on page 45 of WO 98/09948;
   then it was incubated for 24 hours
   IL-6 and TGF-β released in cell supernatant was measured by ELISA assay, taking as 100% release that of placebo group.

The results obtained are reported in Table 4.

The Table shows that both NO-flurbiprofen and NO-indomethacin inhibit the relase of IL-6 and potentiate the release of TGF-β.

### EXAMPLE F5

Effect of Flurbiprofen, NO-Flurbiprofen, Ibuprofen, NO-Ibuprofen on Human Chondrocytes and Proteoglycan Synthesis (In Vitro Study)

Human chondrocytes were isolated by collagenase digestion from knee cartilage collected from patients undergoing knee replacement surgery. Only primary culture was used to avoid phenotype change of human chondrocytes. TNFα (80 ng/ml) was added to all but control cells. Test compounds were dissolved at a concentration 0.02% (w/v) in DMSO (vehicle).

The following compounds were tested:
Flurbiprofen;
NO-flurbiprofen, prepared as described in ex. F1;
Ibuprofen;
NO-ibuprofen, prepared as described in ex. F1.

The test compounds were incubated with cells at a 100 μM concentration for 24 hours.

Cell proliferation was determined by measuring [³H]-thymidine incorporated into newly synthesized DNA. Cell viability was assessed by MTS assay kit.

Proteoglycan synthesis was determined by [³⁵S]-sulfate incorporation. Cells and supernatant were extracted with 4M guanidinium chloride and purified by Sephadex colums chromatography. The amount of [³⁵S]-sulfate was measured by liquid scintillation counter. Results were normalized by the amount of Dna in the sample and expressed as CPM/μg DNA (CPM=count per minute).

The results are reported in Table 5 and are expressed as % cell growth/proteoglycan synthesis with respect to the control group.

The Table shows that NO-flurbiprofen and NO-ibuprofen reversed the decrease of cell proliferation induced by TNFα. No effect on cell viability was found. Both NO compounds reversed the decrease in proteoglycan synthesis induced by TNFα. The parent NSAIDs did not affect TNFα-induced effects on cell proliferation and proteoglycan synthesis. In both experiments the activity of the parent compounds was almost the same as that of the vehicle.

### EXAMPLE F6

Effect of Flurbiprofen, NO-Flurbiprofen, Paracetamol and NO-Paracetamol on the Expression of TGF-β Type II Receptor.

Type II collagen and TGF-β type II receptor (TβRII) expression have been reported as agents playing a crucial role in osteoarthritis (OA) physiopathology. Indeed, in experimental models of OA it was found that the physiological levels of said agents are dramatically decreased. This could be one of the main reasons why OA cartilage erosion continues irreversibly (Osteoarthritis and Cartilage, 1998, 6, 146-149).

The steady-state levels of mRNA for type II collagen and TGF-β type II receptor (TβRII) was evaluated in human articular chondrocytes (HAC), cultured in hypoxia (5% v/v O₂). The cells were treated or not with interleukin-1β (IL-1β) an agent favouring OA pathology, and NO-NSAIDs, or the corresponding NSAIDs at 10⁻⁵M for 48 h.

The following compounds were tested:
flurbiprofen;
NO-flurbiprofen, prepared as described in ex. F1;
Paracetamol;
NO-paracetamol, prepared as described in ex. F1.

It was found that NO-flurbiprofen increased type II collagen mRNA levels (more than 100%) whereas flurbiprofen had no significant effect.

Furthermore NO-paracetamol and NO-flurbiprofen strongly increased TβRII (more than 100%) whereas their corresponding NSAIDS had no effect.

The nitrooxy derivatives according to the present invention stimulate the expression of TGF-β receptor type II and therefore delay the onset or evolution of OA.

TABLE 1
Stimulation of the TGFβ1 production in cellular chondrocyte
cultures to which the compounds mentioned below have been added.
The results are expressed in percentage of TGFβ1 produced in
the samples treated with respect to the un-treated control.

| Compound | % of produced TGFβ1 |
|---|---|
| Controls | 100 |
| NO-Aspirin | 600 |
| Aspirin (comp) | 150 |
| NO-Flurbiprofen | 650 |
| Flurbiprofen (comp) | 120 |
| NO-Diclofenac | 550 |
| NO-Ibuprofen | 700 |
| NO-Paracetamol | 350 |

TABLE 2
Evaluation of the hepatic tolerability (AST, ALT and bilirubin
concentration) in consequence of the administration to rats
of NO-paracetamol and paracetamol. The reported values are
expressed in % with respect to those of the controls

| | AST | ALT | Bilirubin |
|---|---|---|---|
| Treatment | % | % | % |
| Carrier | 100 | 100 | 100 |
| Paracetamol (comp) | 330 | 171 | 200 |
| NO-paracetamol | 160 | 57 | 136 |

TABLE 3
Example F3: effect of flurbiprofen and NO-flurbiprofen on IL-6
release in human monocytes. Results are given as % in the confront
of IL-6 release obtained in the placebo group.

| | IL-6 release |
|---|---|
| Treatment | % in the confront of placebo |
| Placebo | 100 |
| Flurbiprofen (comp) | 100 |
| NO-flurbiprofen | 10 |

TABLE 4
Example F4: effect of flurbiprofen, NO-flurbiprofen,
indomethacin, NO-indomethacin on IL-6 and TGF-βrelease
in mouse spleen lymphocytes.

| | IL-6 release | TGF-β release |
|---|---|---|
| | % in the confront | % in the confront |
| Treatment | of placebo | of placebo |
| Placebo | 100 | 100 |
| Flurbiprofen (comp) | 100 | 115 |
| NO-flurbiprofen | 10 | 150 |
| Indomethacin (comp) | 90 | 70 |
| NO-indomethacin | 20 | 130 |

TABLE 5
Example F5: effect of flurbiprofen, NO-flurbiprofen, ibuprofen,
NO-ibuprofen on cell proliferation and proteoglycan synthesis.

| | Cell proliferation | Proteoglycan synthesis |
|---|---|---|
| | % in the confront | % in the confront |
| Treatment | of control | of control |
| Control | 100 | 100 |
| Vehicle | 50 | 22 |
| Flurbiprofen (comp) | 53 | 26 |
| NO-flurbiprofen | 90 | 70 |
| Ibuprofen (comp) | 48 | 24 |
| NO-ibuprofen | 95 | 55 |

1. Use for the preparation of disease-modifying drugs drugs for the prevention and treatment of arthritis therapy of compounds or salts thereof having the following general formula:
A-(B)_{b0}—(C)_{c0}—N(O)_{S} (I)
wherein:
s is an integer and is equal to 1 or 2, preferably 2;
c0 is an integer and is equal to 0 or 1;
b0 is an integer and is 0 or 1; with the proviso that at least one between c0 and b0 is different from zero;
A=R-T₁-, wherein
R— is the radical of a non steroidal antiinflammatory precursor drug excluding the compounds having 2-oxo-1H-indolic structure, or the radical of a non steroidal antiinflammatory/analgesic drug;
T₁=(CO)ₜ or (X)_{t′}, wherein X=—O—, —S—, —N(R_{1C})—, R_{1C} is H or C₁-C₅ linear or branched alkyl, t and t′ are integers and equal to zero or 1, with the proviso that t=1 when t′=0; t=0 when t′=1;
B=-T_{B}-X₂-T_{BI}- wherein
T_{B} and T_{BI} are equal or different;
T_{B}=(CO) when the reactive function in the precursor drug is —OH or —NH(R_{1C}); T_{B}=X, as above, when the reactive function in the precursor drug is —COOH;
T_{BI}=(CO)ₜₓ or (X)ₜₓₓ, wherein tx and txx have the value of 0 or 1; with the proviso that tx=1 when txx=0, tx=0 when txx=1; X is as above;
X₂ is a bivalent linking group as defined below;
C is the bivalent radical -T_{C}-Y— wherein
when b0=c0=1: T_{C}=(CO) when tx=0, T_{C}=X when txx=0, X being as above;
when b0=0: T_{C}=(CO) when t=0, T_{C}=X when t′=0, X being as above;
when c0=0: tx=0, T_{BI}=X=—O—.
Y is:
Yₚ: 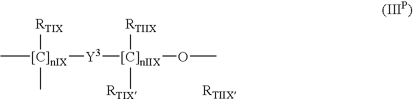
wherein:
nIX is an integer from 0 to 10, preferably from 1 to 3;
nIIX is an integer from 1 to 10, preferably from 1 to 3;
R_{TIX}, R_{TIX′}, R_{TIIX}, R_{TIIX′}, equal to or different from each other are H or C₁-C₄ linear or branched alkyl; preferably R_{TIX}, R_{TIX′}, R_{TIIX}, R_{TIIX′} are H.
Y³ is a saturated, unsaturated or aromatic heterocyclic ring containing one or two nitrogen atoms having 5 or 6 atoms,
or Y can be:
Y₀, selected from the following:
a —R′O— alkylenoxy group wherein R′ is linear or branched when possible C₁-C₂₀, preferably having from 2 to 6 carbon atoms, or a cycloalkylene having from 5 to 7 carbon atoms, in the cycloalkylene ring one or more carbon atoms can be substituted by heteroatoms, the ring can have side chains of R′ type, R′ being as above; or one of the following groups: 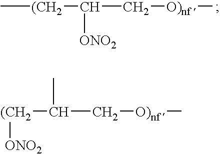
wherein nf′ is an integer from 1 to 6 preferably from 1 to 4; 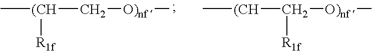
wherein R_{1f}=H, CH₃ and nf′ is an integer from 1 to 6;
preferably from 1 to 4;
or Y is Y_{Ar} and is selected from the following: 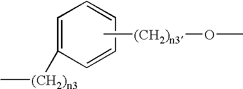
wherein n3 is an integer from 0 to 3 and n3′ is an integer from 1 to 3; 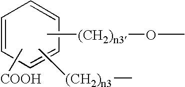
wherein n3 and n3′ have the above meaning;
X₂, bivalent radicalm is such that the corresponding precursor of B, -T_{B}-X₂-T_{BI}- wherein the free valences of T_{B} and of T_{BI} are saturated each with OZ, with Z or with —N(Z^{I})(Z^{II}), wherein Z=H, C₁-C₁₀, preferably C₁-C₅ linear or branched when possible alkyl, Z^{I}, Z^{II} equal or different have the Z values as above, depending on that T_{B} and/or T_{BI}=CO or X, in function of the values of t, t′, tx and txx;
the precursor of B is selected from the following:
aminoacids,
hydroxyacids,
aromatic and heterocyclic mono- and polyalchols,
compounds containing at least one free acid function.

2. Use according to claim 1, wherein the precursor of B is selected from the following:—aminoacids selected from the following: L-carnosine (formula CI), anserine (CII), selenocysteine (CIII), selenomethionine (CIV), penicillamine (CV), N-acetylpenicillamine (CVI), cysteine (CVII), N-acetylcysteine (CVIII), glutathione (CIX) or esters thereof, preferably ethyl or isopropyl ester: 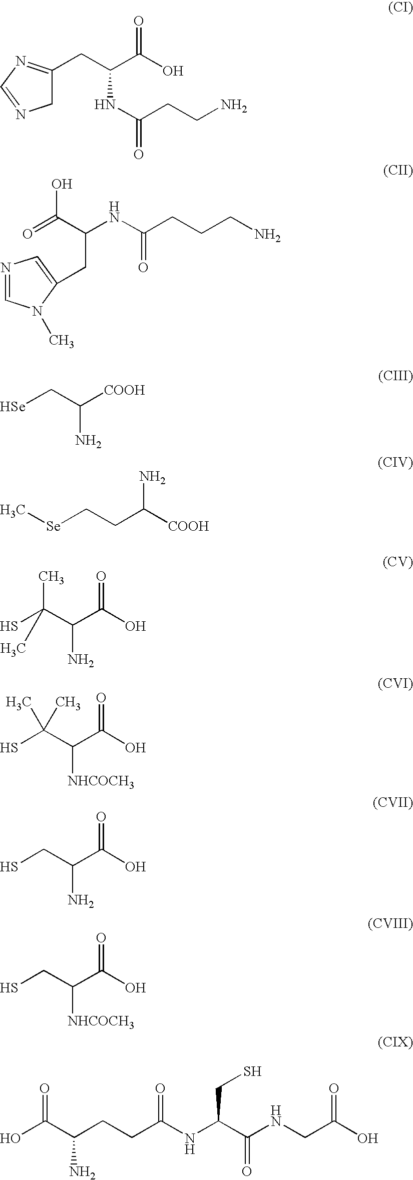
hydroxyacids, selected from the following: gallic acid (formula DI), ferulic acid (DII), gentisic acid (DIII), citric acid (DIV), caffeic acid (DV), dihydrocaffeic acid (DVI), p-cumaric acid (DVII), vanillic acid (DVIII): 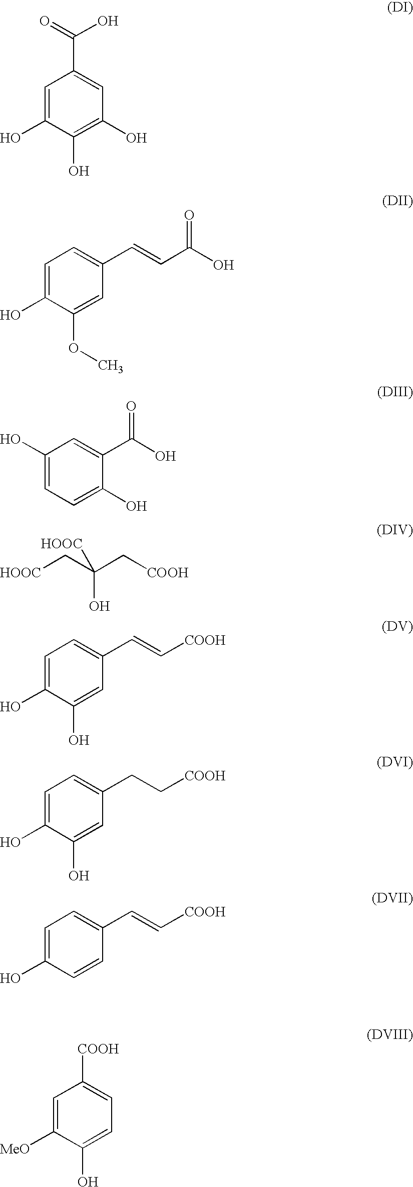
aromatic and heterocyclic mono- and polyalcohols, selected from the following: nordihydroguaiaretic acid (EI), quercetin (EII), catekin (EIII), kaempferol (EIV), sulphurethyne (EV), hydroquinone (EVIII), gossypol (EIX), reductic acid (EX), methoxyhydroquinone (EXI), hydroxyhydroquinone (EXII), propyl gallate (EXIII), 3,5-di-ter-butyl-4-hydroxybenzyl-thioglycolate (EXXIV), allopurinol (EXXXI); saccharose (EC), ascorbic (ECI) and isoascorbic acid (ECII), p-cumaric alcohol (ECIII), 4-hydroxy-phenylethylalcohol (ECIV), coniferyl alcohol (ECV): 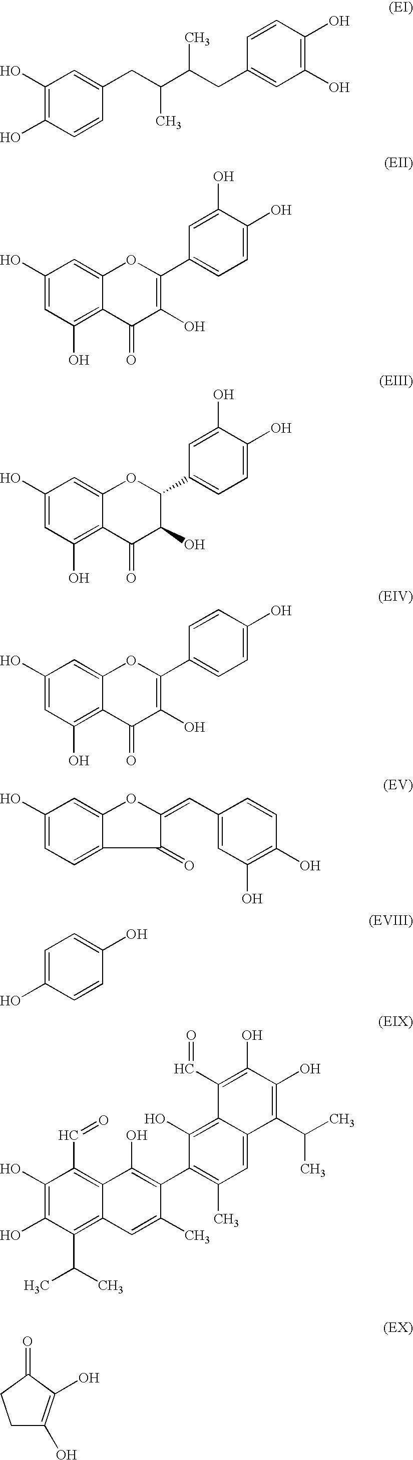 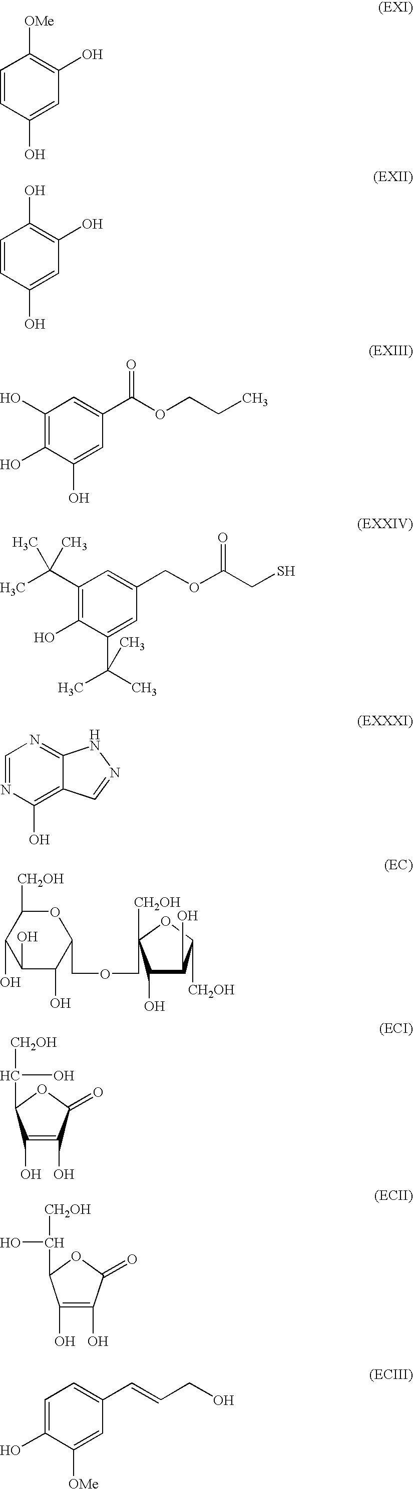 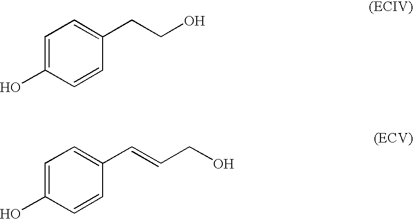
compounds containing at least one free acid function, selected from the following: 3,3′-thiodipropionic acid (NI), fumaric acid (NII), dihydroxymaleic acid (NIII), edetic acid (NV):

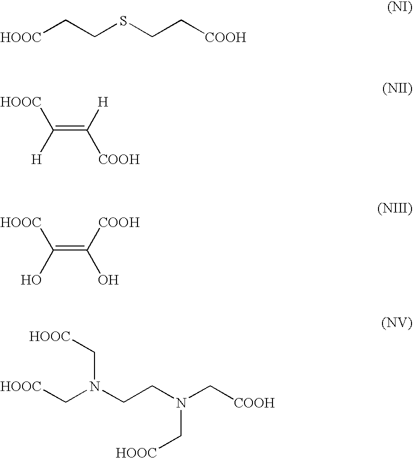

3. Use according to claim 1, wherein in the compounds of formula (I) when b0=c0=1, the bonds between the drug radical and X₂ and between X₂ and Y are, independently the one from the other, of ester, thioester, amide type;
when b0=0 and c0=1 the bond between the drug radical and Y is of ester, thioester, amide type.

4. Use according to claim 1, wherein the R radical is selected from the following groups: 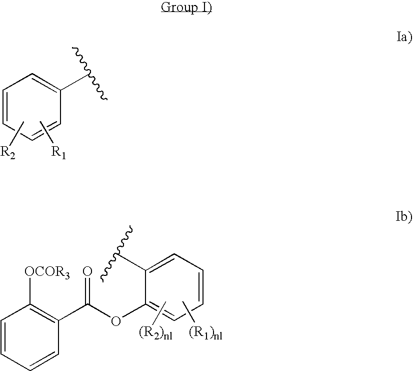
wherein:
R₁ is H or —OCOR₃; wherein R₃ is methyl, ethyl or C₃-C₅ linear or branched alkyl, or the residue of an heterocycle with only one ring having 5 or 6 atoms partially or totally hydrogenated, or aromatic, containing one or more heteroatoms independently selected from O, N and S;
R₂ is hydrogen, hydroxy, halogen, C₁-C₄ linear or branched alkyl, C₁-C₄ linear or branched alkoxyl; a C₁-C₄ linear or branched perluoroalkyl, for example trifluoromethyl; nitro, amino, mono- or di-(C₁₋₄)alkylamino;
with the proviso that in formula Ia) R₁ and R₂ are not contemporaneously H;
preferably when R₁=H R₂=OH;
preferably in the compounds of formula Ia) T₁=—CO— and:
R₁=acetoxy, preferably in ortho position with respect to —CO—, R₂ is hydrogen; in this case the formula Ia) represents the acetylsalicylic acid residue;
R₁=H R₂=OH, preferably in ortho position with respect to —CO—, in this case the formula Ia) represents the salicyilic acid residue;
in formula Ib) nI is an integer 0 or 1;
preferably in the compounds of formula Ib) R₃=CH₃, nI=0, T₁=—CO—; in this case Ib) is the acetylsalicylsalicylic acid residue; 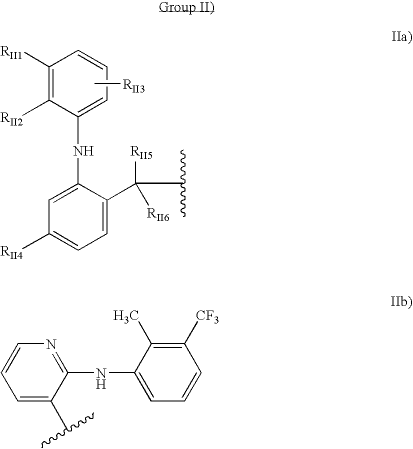
wherein:
R_{II5} is H, C₁-C₃ linear or branched when possible alkyl;
R_{II6} has the same meaning as R_{II5}, or when R_{II5} is H it is benzyl;
R_{II1}, R_{II2} and R_{II3} are independently hydrogen, C₁-C₆ linear or branched alkyl, or C₁-C₆ linear or branched alkoxy, or Cl, F, Br;
R_{II4} is R_{II1} or bromine;
the compounds are preferred wherein R_{II1}, R_{II4} are hydrogen and R_{II2} and R_{II3} are chlorine in ortho position with respect to NH; R_{II5} and R_{II6} are H, T₁=—CO—, when the free valence is saturated with OH the precursor compound is known as diclofenac.
IIb) is the residue of the 2-[(2-methyl-3-(trifluoro methyl)phenyl]amino]-3-pyridincarboxylic] acid when T₁=—CO— and the free valence is saturated with OH the compound is known as flunixin; 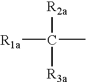
wherein:
R₂ₐ and R₃ₐ are H, C₁-C₁₂ linear or branched, substituted or not, alkyl or allyl, with the proviso that when one of the two is allyl the other is H;
preferably R₂ₐ and R₃ₐ, equal or different, are H, C₁-C₄ alkyl;
R₁ₐ is selected from: 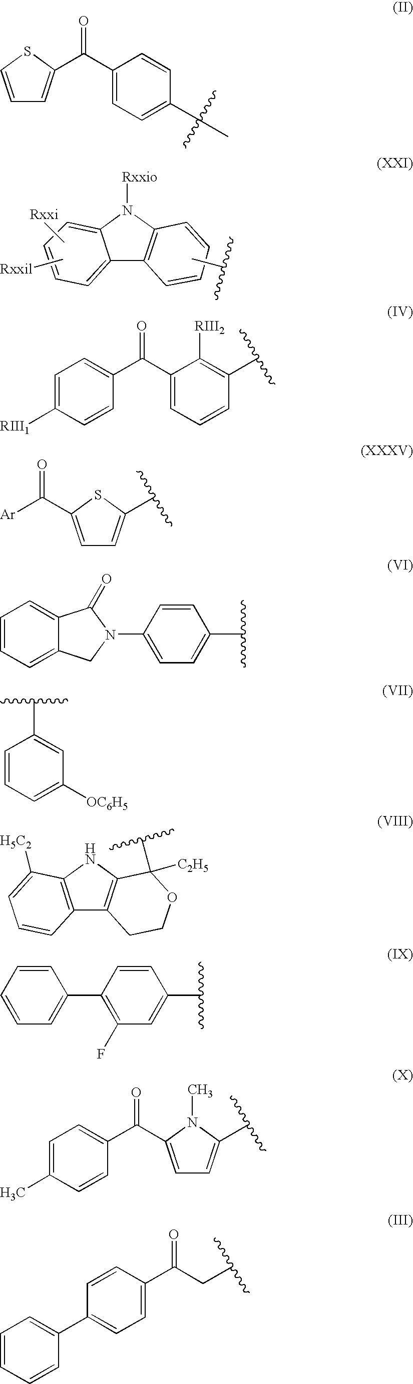
IIID) R₁ₐ corresponds to the following formulas: 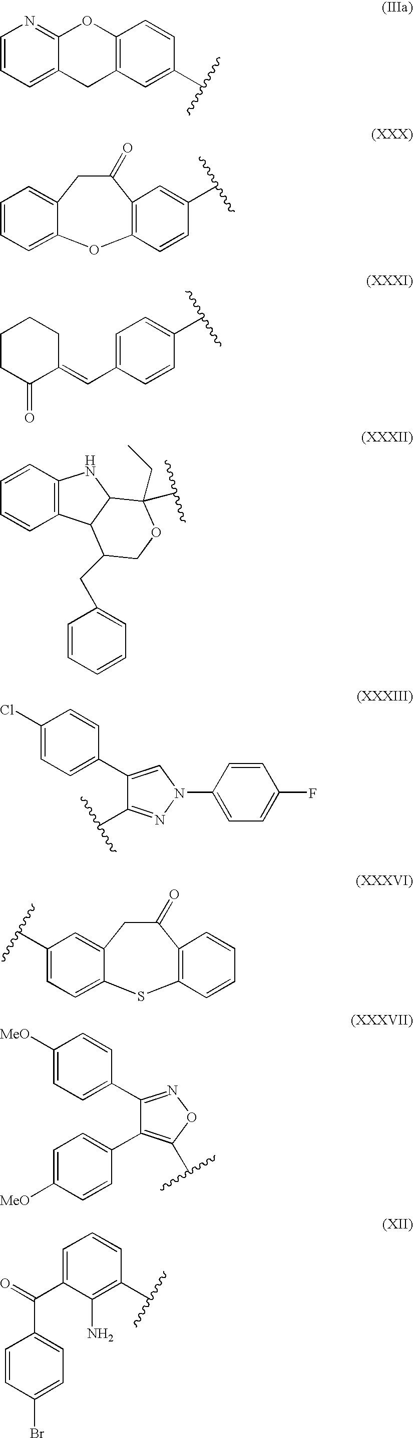 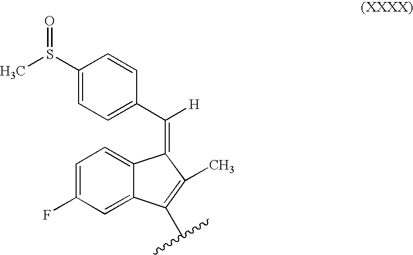
wherein the meanings are the following:
when R₁ₐ is as defined in formula (IV), Ketoprofen residue:
R_{II1} is H, SR_{III3} wherein R_{III3} is C₁-C₄ linear or branched alkyl;
R_{III2} is H, hydroxy;
the compounds wherein R_{III1} and R_{III2} are H, R₃ₐ is H, and R₂ₐ is methyl, T₁=—CO— are preferred;
when R₁ₐ is as defined in formula (XXI), carprofen residue:
Rₓₓᵢₒ is H, alkyl from 1 to 6 C atoms linear or branched, C₁-C₆ alkoxycarbonyl linked to a C₁-C₆ alkyl, C₁-C₆ carboxyalkyl, C₁-C₆ alkanoyl, optionally substituted with halogens, benzyl or halobenzyl, benzoyl or halobenzoyl;
Rₓₓᵢ is H, halogen, hydroxy, CN, C₁-C₆ alkyl containing or not containing OH groups, C₁-C₆ alkoxy, acetyl, benzyloxy, SRₓₓᵢ₂ wherein Rₓₓᵢ₂ is C₁-C₆ alkyl; C₁-C₃ perfluoroalkyl; C₁-C₆ carboxyalkyl containing or not containing OH groups, NO₂, amino; sulphamoyl, dialkyl sulphamoyl with C₁-C₆ alkyl, or difluoroalkylsulphonyl with C₁-C₃ alkyl;
Rₓₓᵢ₁ is halogen, CN, C₁-C₆ alkyl containing one or more OH groups, C₁-C₆ alkoxy, acetyl, acetamido, benzyloxy, SR_{III3} being R_{III3} as above, C₁-C₃ perfluoroalkyl, hydroxy, C₁-C₆ carboxyalkyl, NO₂, amino, C₁-C₆ mono- or di-alkyl-amino; sulphamoyl, C₁-C₆ di-alkylsulphamoyl, or di-fluoroalkylsulphamoyl as above; or Rₓₓᵢ together with Rₓₓᵢ₁ is a C₁-C₆ alkylen-dioxy;
the compounds are preferred wherein Rₓₓᵢₒ is H, the linking group is in position 2, Rₓₓᵢ₁ is H, Rₓₓᵢ₁ is chlorine and is in para position with respect to the nitrogen;
R₃ₐ is H, R₂ₐ is methyl and T₁=—CO—;
when R₁ₐ is as defined in formula (XXXV) tiaprofenic acid residue:
Ar is phenyl, hydroxyphenyl optionally mono- or polysubstituted with halogen, alkanoyl and C₁-C₆ alkoxy, C₁-C₆ trialkyl, preferably C₁-C₃, cyclopentyl, cyclohexyl, cycloheptyl, heteroaryl, preferably thienyl, furyl containing or not containing OH, pyridyl;
the preferred compounds of (XXXV) are those wherein Ar is phenyl, R₃ₐ is H, R₂ₐ is methyl and T₁=—CO—;
when R₁ₐ is as defined in formula (II), suprofen residue, R₃ₐ is H, R₂ₐ is methyl and T₁=—CO—;
when R₁ₐ is as defined in formula (VI), R is the residue of indoprofen when T₁=—CO—, R₂ₐ=H and R₃ₐ=CH₃; of indobufen when R₂ₐ is equal to H and R₃ₐ=C₂H₅; T₁=—CO—;
when R₁ₐ is as defined in formula (VIII), R is the etodolac residue when R₂ₐ=R₃ₐ=H and T₁=—CO—;
when R₁ₐ is as defined in formula (VII), R is the fenoprofen residue when R₃ₐ=H, R₂ₐ=CH₃ and T₁=—CO—;
when R₁ₐ is as defined in formula (III), R is the fenbufen residue when R₂ₐ=R₃ₐ=H and T₁=—CO—;
when R₁ₐ is as defined in formula (IX), R is the flurbiprofen residue when R₃ₐ=H, R₂ₐ=CH₃, T₁=—CO—;
when R₁ₐ is as defined in formula (X) R is the tolmetin residue when R₂ₐ=R₃ₐ=H, T₁=—CO—.
In group IIID) R₁ₐ corresponds to the following formulas:
IIIa), when R₂ₐ=H and R₃ₐ=CH₃ the pranoprofen residue is obtained: α-methyl-5H-[1]benzopyran-[2,3-b]pyridin-7-acetic acid; in the preferred compound R₂ₐ=H, R₃ₐ=CH₃, T₁=—CO— and in the precursor the free valence is saturated with OH;
(XXX), when R₂ₐ=H and R₃ₐ=CH₃ the bermoprofen residue is obtained: dibenz[b,f]oxepin-2-acetic acid; in the preferred compound R₂ₐ=H, R₃ₐ=CH₃, T₁=—CO—;
(XXXI), when R₂ₐ=H and R₃ₐ=CH₃, R is the radical of the compound CS-670: 2-[4-(2-oxo-1-cyclohexyliden methyl)phenyl]propionic acid; the preferred compound has R₂ₐ=H, R₃ₐ=CH₃, T₁=—CO—;
(XXXII), when R₂ₐ=R₃ₐ=H, the pemedolac residue is obtained;
when R₂ₐ=R₃ₐ=H T₁=—CO—;
(XXXIII), when R₂ₐ=R₃ₐ=H, the pirazolac residue is obtained: 4-(4-chlorophenyl)-1-(4-fluorophenyl)-3-pyrazol acid derivatives;
the preferred compounds have R₂ₐ=R₃ₐ=H, T₁=—CO—;
(XXXVI), when R₂ₐ=H, R₃ₐ=CH₃ the zaltoprofen residue is obtained; when the residue is saturated with an hydroxyl or aminic group, or with the carboxylic function the compounds are known as dibenzotiepin derivatives; in the preferred compounds R₂ₐ=H, R₃ₐ=CH₃, T₁=—CO—;
(XXXVII), when R₂ₐ=R₃ₐ=H the mofezolac residue is obtained: 3,4-di(p-methoxyphenyl)isoxazol-5-acetic acid when the residue is CH₂—COOH; in the preferred compounds R₂ₐ=R₃ₐ=H, T₁=—CO—;
(XII), when R₂ₐ=R₃ₐ=H the bromfenac residue is obtained: 2-amino-3-(4-bromobenzoyl)benzeneacetic acid; the preferred compounds have T₁=—CO—, R₂ₐ=R₃ₐ=H;
(XXXX) when R₂ₐ=R₃ₐ=H the sulindac residue is obtained: (Z)-5-fluoro-2-methyl-1-[[4-(methyl sulphinyl)-phenyl]methylene]-1H-inden-3-acetic aid; the preferred compounds have T₁=—CO—, R₂ₐ=R₃ₐ=H;
in Group IV) R is 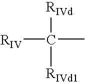
wherein:
R_{IVd} and R_{IVd1} are at least one H and the other an alkyl from C₁ to C₆ linear or branched, preferably C₁-C₂, or difluoroalkyl with C₁-C₆ alkyl, C₁ preferred, or R_{IVd} and R_{IVd1} form together a methylene group;
R_{IV} has the following meaning; 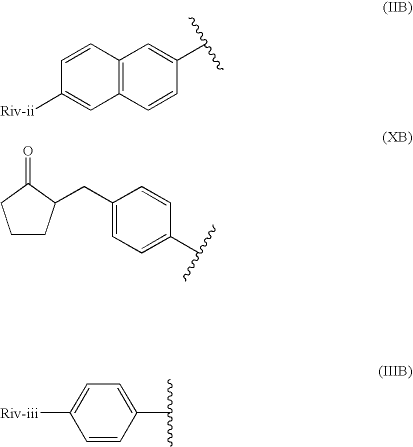
wherein the compounds of group IV) have the following meanings:
in formula (IIB):
R_{IV-ii} is C₁-C₆ alkyl, C₃-C₇ cycloalkyl, C₁-C₇ alkoxymethyl, C₁-C₃ trifluoroalkyl, vinyl, ethynyl, halogen, C₁-C₆ alkoxy, difluoroalkoxy with C₁-C₇ alkyl, C₁-C₇ alkoxymethyloxy, alkylthiomethyloxy with C₁-C₇ alkyl, alkyl methylthio with C₁-C₇ alkyl, cyano, difluoromethylthio, phenyl- or phenylalkyl substituted with the C₁-C₈ alkyl; preferably R_{iV-ii} is CH₃O—, R_{IVd} is H and R_{IVd1} is CH₃, and is known as naproxene residue; T₁=—CO—;
in formula (XB), of which the loxoprofen residue has been indicated, the compounds wherein R_{IVd} is H and R_{IVd1} is CH₃, T₁=—CO— are preferred;
in formula (IIIB):
R_{iV-iii} is a C₂-C₅ branched or not branched alkyl, C₂ and C₃ alkyloxy, allyloxy, phenoxy, phenylthio, cycloalkyl from 5 to 7 C atoms, optionally substituted in position 1 by a C₁-C₂ alkyl;
the compound is preferred wherein R_{iV-iii} is 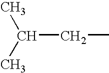
and R_{IVd}=H, R_{IVd1} is CH₃, compound known as ibuprofen residue, T₁=—CO—; 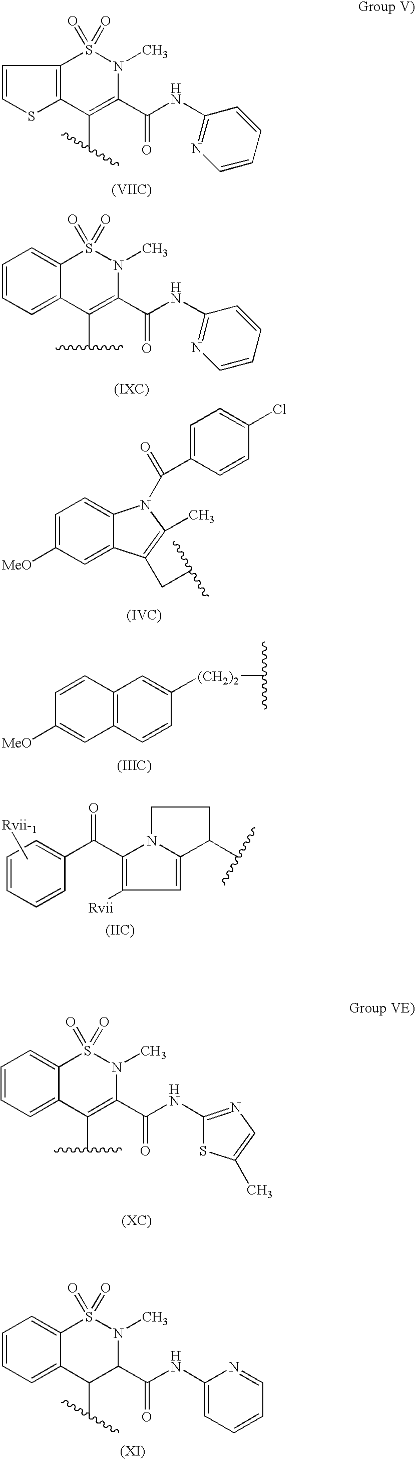 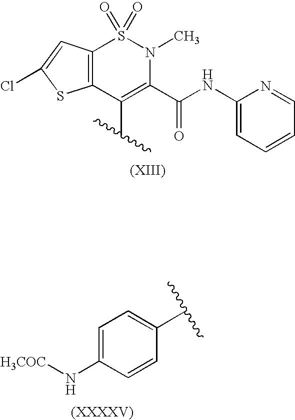
In group V), the compounds have the following meanings:
when R is the formula (IIC),
R_{Vii} is H or a C₁-C₄ linear or branched alkyl;
R_{Vii-1} is R_{Vii}, or C₁-C₄ linear or branched alkoxy; Cl, F, Br; the position of R_{Vii-1} being ortho, or meta, or para;
the Ketorolac residue is preferred, wherein R_{Vii} and R_{Vii-1} are H, and T₁=—CO—;
when R is the formula (VIIC),
of which the tenoxicam residue has been indicated, T₁=—O—;
when R is the formula (IXC), wherein T₁=—O—, the piroxicam residue has been indicated;
when R is the formula (IIIC),
wherein T₁=—CO—, of which the nabumetone residue has been indicated;
when R is the formula (IVC),
wherein T₁=—CO—, of which the indomethacin residue has been indicated;
when R is the formula (XC), the residue X is known as meloxicam;
the preferred compounds are those in which T₁=—CO—;
when R is the formula (XI) the residue is known as ampiroxicam when the termination is —CH(CH₃)OCOC₂H₅; the preferred compounds have T₁=—CO—;
when R is the formula (XIII) and the valence is saturated with H, the residue derives from lornoxicam; the preferred compounds have T₁=—O—;
when R is the formula (XXXXV), T₁=—O— and the valence is saturated with H, the compound known as paracetamol is obtained.

5. Use according to claim 1, wherein in the compounds of formula (I) Y³ of formula (III^{P}) of C is selected from the following bivalent radicals:

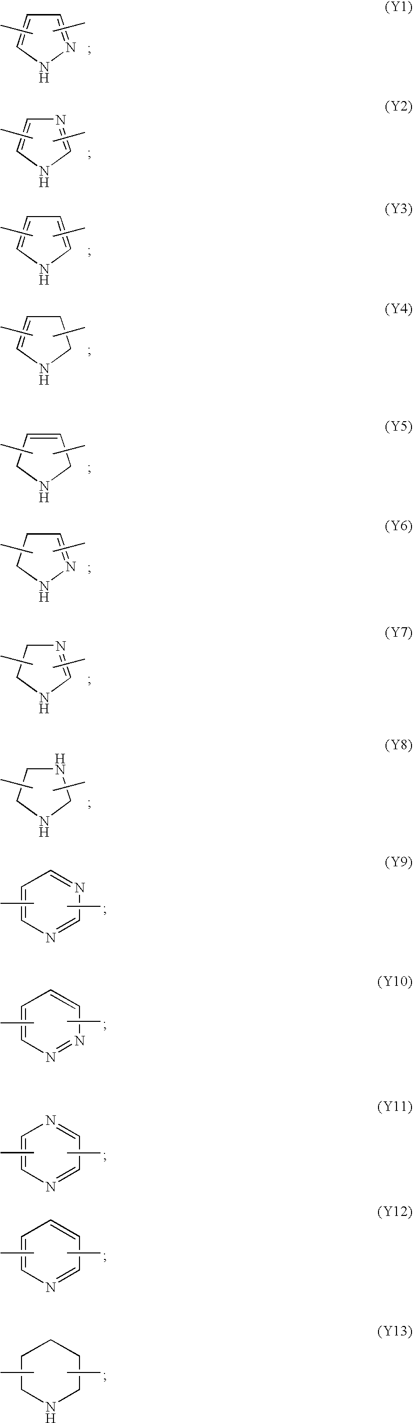

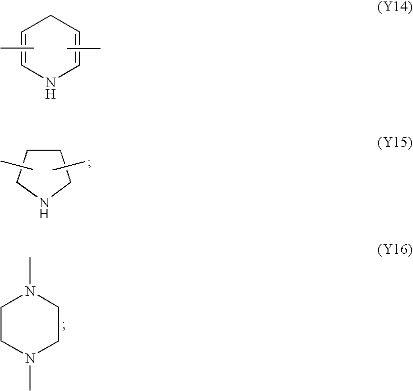

6. Use according to claim 5, wherein Y³ is selected from the following: (Y12) with the two free valences in the ortho positions with respect to the nitrogen atom; (Y16) with the two valences linked to the two heteroatoms, Y1 (pyrazol) 3,5-disubstituted; Y16 is particularly preferred.

7. Use according to claim 1, wherein the following compounds are used:
2-acetyloxybenzoic acid 3-nitrooxymethyl phenyl ester (I^{C});
2-fluoro-alpha-methyl[1,1′-biphenyl]-4-acetic acid 4-ni-trooxy butylester (II^{C});
2-[(2,6-dichlorophenyl)amino]benzenacetic acid 4-ni-trooxy butyl ester (III^{C});
(S)-N-acetyl-[alpha-methyl-4-(2-methylpropyl)benzen-acetyl]cysteine 4-nitrooxybutylester having formula: 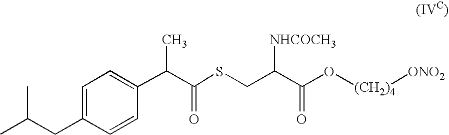
4-nitrooxybutanoic acid 4-acetylaminophenylester (V^{C});
trans-3-[4-[2-fluoro-alpha-methyl(1,1′-biphenyl)-4-acetyloxy]-3-methoxyphenyl]-2-propenoic acid 4-(nitrooxy)butyl ester, having formula: 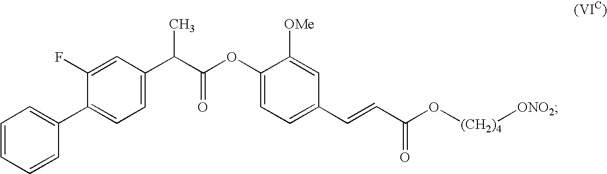
2-Fluoro-alpha-methyl[1,1′-biphenyl]-4-acetic acid 3-(ni-trooxymethyl)phenyl ester having formula: 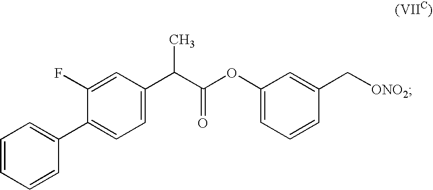
(S)-N-acetyl-[2-fluoro-alpha-methyl(1,1′-biphenyl)-4-acetyl]cysteine 4-(nitrooxy)butyl ester having formula: 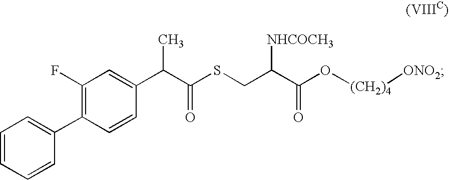
2-Fluoro-alpha-methyl[1,1′-biphenyl]-4-acetic acid 6-(nitrooxy methyl)-2-methylpyridyl ester having formula 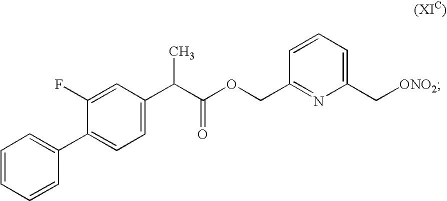
(S)-6-methoxy-alpha-methyl-2-naphthalenacetic acid 4-(nitrooxy)butyl ester having formula: 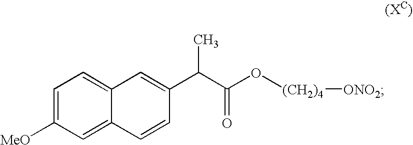
(S)-6-methoxy-alpha-methyl-2-naphthalenacetic acid 3-(nitrooxymethyl)phenyl ester having formula: 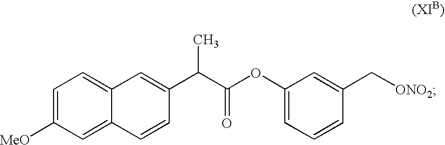
(S)-6-methoxy-alpha-methyl-2-naphthalenacetic acid 6-(nitrooxymethyl)-2-methylpyridyl ester having formula: 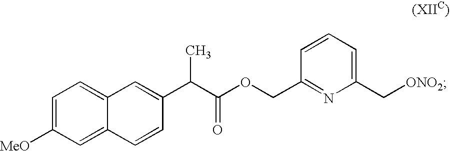
trans-3-[4-[6-methoxy-alpha-methyl-2-naphthalenacetyl oxy]-3-methoxyphenyl]-2-propenoic acid 4-(nitrooxy)butyl ester having formula: 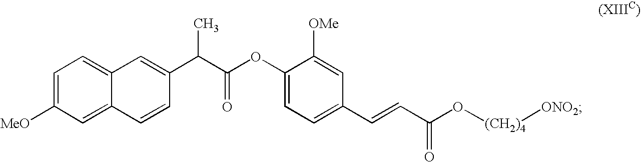
(S,S)-N-acetyl-S-(6-methoxy-alpha-methyl-2-naphthaleneacetyl)cysteine 4-(nitrooxy)butyl ester having formula: 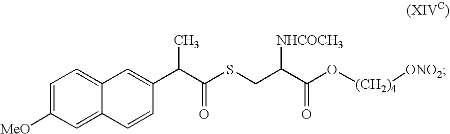
2-[(2,6-dichlorophenyl)amino]benzenacetic acid 4-(nitrooxy methyl)phenylmethyl ester having formula: 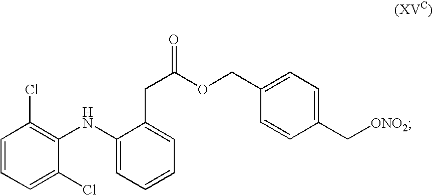
2-[(2,6-dichlorophenyl)amino]benzenacetic acid 6-(nitrooxymethyl)-2-methylpyridyl hydrochloride ester having formula: 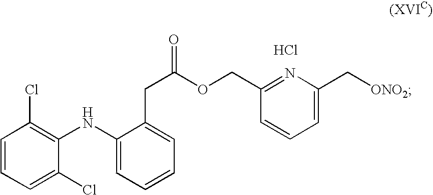
(S)-3-benzoyl-alpha-methyl-benzenacetic acid 4-(nitro oxybutyl)ester having formula: 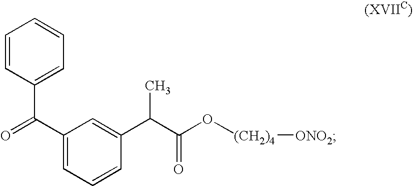
(S)-3-benzoyl-alpha-methyl-benzenacetic acid 3-(nitro oxypropyl)ester having formula: 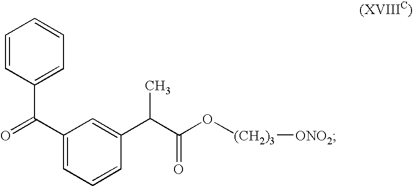
(S)-3-benzoyl-alpha-methyl-benzenacetic 4-(nitro oxymethyl)phenylmethyl ester having formula: 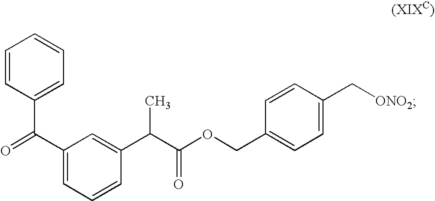
5-benzoyl-2,3-dihydro-1H-pyrrolizine-1-carboxylic acid 4-(nitrooxy)butyl ester having formula: 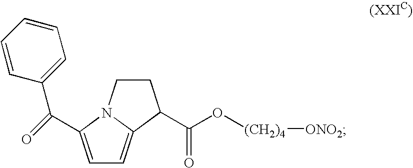
2-[(2,6-dichlorophenyl)amino]benzenacetic acid 5 (nitro oxy)ethyloxyethyl ester having formula: 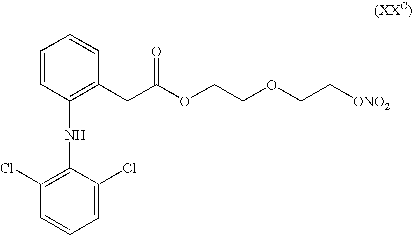
1-(4-Chlorobenzoyl)-5-methoxy-2-methyl-1H-indole-3-acetic acid 3-(nitrooxymethyl)phenyl ester (XXI^{C})

8. Use according to claim 1, wherein the compounds of formula (I) are administered in pharmaceutical formulations by oral, parenteral and topical administration.

9. Use according to claim 1 for the prevention of arthritis relapses.
